(19) United States
(12) Patent Application Publication
(10) Pub. No.: US 2011/0318286 A1
(43) Pub. Date: Dec. 29, 2011

(54) SPF ENHANCED EXTENDED COLOR BULK POWDERS AND METHODS OF MAKING THEREOF

(75) Inventors: Yoshiaki Kawasaki, Woodstock, CT (US); Katsumi Shimizu, Pomfret Center, CT (US); Noriaki Yamamoto, Irvine, CA (US); Mark George LaPage, Dudley, MA (US)

(73) Assignee: US Cosmetics Corporation, Dayville, CT (US)

(21) Appl. No.: 13/230,149

(22) Filed: Sep. 12, 2011

Related U.S. Application Data
(63) Continuation-in-part of application No. 12/115,901, filed on May 6, 2008.
(60) Provisional application No. 60/928,146, filed on May 7, 2007.
(60) Provisional application No. 61/518,785, filed on May 12, 2011.

(51) Int. Cl. A61K 8/36; A61Q 19/00; A61Q 17/04

(52) U.S. Cl. 424/60; 424/59

(57) ABSTRACT

This application discloses, among other things, extended color bulk powders and partially and dispersions and methods of making thereof. Fully and partially extended color powders and fully and partially extended color dispersions can be used in cosmetic and makeup products, personal care products, and pharmaceutical products.

### RELATED APPLICATIONS

This application is a continuation-in-part of application Ser. No. 12/115,901, filed May 6, 2008, which claims the priority of provisional Application No. 60/928,146, filed May 7, 2007, and of International Application No. PCT/US2010/038966, filed Jun. 17, 2010, which claims the priority of provisional Application No. 61/218,785, filed Jun. 19, 2009. Each application is herein incorporated by reference in its entirety for all purposes.

### FIELD

This application relates to fully extended color powder materials and dispersions, and methods of making fully extended color powder materials and dispersions useful for cosmetic products such as foundations, eye shadows, lip colors, mascaras, lotions, and creams.

This application also relates generally to cosmetic formulations having enhanced UV protection factors. The present application relates particularly, but not by way of limitation, to UV-protecting cosmetic formulations comprising cosmetic powders and having low loadings of organic sunscreens.

### INTRODUCTION

Current cosmetics and make-ups on market employ hydrophobically modified pigments and substrates as components for establishing excellent resistance against sebum and sweat. The conventional manufacturing process for preparing cosmetic and make-up products includes mixing pigments, extenders and/or other substrates and then atomizing them until the colors are well developed. Oily components and auxiliary materials are added to the mixed powder, and then it is atomized to spread the oily components evenly onto the powder for making powder products. For liquid foundations, the mixed powder is dispersed into oily components by a homogenizer. This process requires many hours to pass the powder components through the atomizer several times to deagglomerate pigments and extend color. If color adjustment is needed for the product, the same procedure has to be done for adding small amounts of color pigments to the formula, which means more processing time is required.

Existing cosmetic and make-up products exhibit, for example, uneven color; color change on the skin after the application (the shade on the skin appears different from the shade of the cosmetic product prior to application); color streaks; low skin adhesion; shade changes on the skin over time (not long lasting); uneven wear or irregular spreadability of the cosmetic or makeup; and easy breakage of the pressed cakes (not strong enough).

Many lotion-type sunscreens are currently available on the market. Typically, sunscreen formulas are water-in-oil (W/O) or oil-in-water (O/W) emulsions or are anhydrous systems. In order to obtain a high sun protection factor (SPF) and particularly a high protection factor relative to UV-A radiation (PFA), sunscreen formulations typically incorporate extensive amounts of oil-based, UV-active materials. The use of large amounts of oil-based, UV-actives causes the texture of the resulting sunscreens to be oily, greasy, and tacky. Oils are also undesirable because they may enhance the transdermal permeation of other formulation ingredients including ingredients for which transdermal administration may be inappropriate. In addition to these undesirable properties, the high loading of oil-based UV-actives often causes adverse skin reactions in sensitive individuals.

Commercial sunscreens are typically formulated to yield about 1 to 2 SPF units per weight percent (wt %) UV-active ingredient. For example, typical SPF 20 sunscreen formulations contain approximately 13% UV-active materials. It is often desirable to formulate sunscreen with much higher SPF ratings. To formulate sunscreens at the higher SPF rating requires corresponding increases in the concentration of oil-based, organic UV-actives.

It is desirable to formulate sunscreens with increasingly high SPF values to confer higher levels of protection. However, the current formulation metric implies higher degrees of unwanted side-effects. If organic UV absorbers are used in formulations at the lowest possible level, tactile issues and safety concerns would be ameliorated. Also, production costs would be lower as lesser amounts of raw materials used in the formulation. Therefore, there exists a need to formulate sunscreens having lower amounts of organic UV-actives.

Other objects and advantages will become apparent from the following disclosure.

### SUMMARY

This application relates to fully and partially extended color bulk powders and bulk dispersions. Fully and partially extended color bulk powders include at least one pigment and at least one substrate, wherein the pigment or substrate has a surface that has been chemically immobilized with at least one surface-treatment agent; and wherein the pigment adheres to the substrate. Fully and partially extended color bulk dispersions include at least one pigment and at least one substrate, wherein the pigment or substrate has a surface that has been chemically immobilized with at least one surface-treatment agent; wherein the pigment adheres to the substrate, and wherein the material is dispersed in a liquid medium.

This application provides an extended color bulk powder. According to some embodiments, the extended color bulk powder comprises at least one pigment, at least one substrate, at least one carboxylate surface-treatment agent, at least one organosilane surface-treatment agent, and at least one lipophilic UV absorber (e.g., wherein each of the at least one surface-treatment agent is chemically immobilized to a surface of the at least one pigment or substrate. According to some embodiments, both the surface of the at least one pigment and the surface of the at least one substrate are treated by the at least one surface-treatment agent.

This application also relates to methods for preparing fully and partially extended color bulk powders and fully and partially extended color bulk dispersions. A method for preparing a fully or partially extended color bulk powder includes providing at least one pigment and a substrate; contacting the substrate or pigment with a surface-treatment agent to produce a surface-modified substrate or pigment material, thereby producing a substrate having adhered thereto the pigment; and blending the material until it is fully or partially extended. A method for preparing a fully or partially extended color bulk dispersion includes providing at least one pigment and a substrate; contacting the substrate or pigment with a surface-treatment agent to produce a surface-modified substrate or pigment material, thereby producing a substrate having adhered thereto the pigment; blending the material until it is fully or partially extended, and dispersing the blended material in a liquid medium.

According to some embodiments, methods are provided for preparing an extended color bulk powder comprising providing at least one pigment; providing at least one substrate; providing at least one UV absorber; contacting the pigment or substrate with at least one carboxylate surface-treatment agent and at least one organosilane surface-treatment agent to produce a surface-modified pigment or substrate, thereby producing a substrate to which the pigment adheres; and blending the resulting material.

The present disclosure relates to a cosmetic formulation comprising at least one organic, UV-active material and at least one cosmetic powder material such that the formulation has an SPF Index of at least 3.0. According to aspects of the disclosure, the cosmetic formulation has an SPF Index of at least 4.0. According to aspects of the disclosure, the cosmetic formulation has an SPF Index of at least 6.0. According to aspects of the disclosure, the cosmetic formulation has an SPF Index of at least 8.0. According to aspects of the disclosure, the cosmetic formulation has an SPF Index of at least 10.0.

According to aspects of the disclosure, the cosmetic formulation comprises at least one organic, UV-active material. According to aspects of the disclosure, the organic UV-active material is any organic sunscreen which absorbs, blocks, or otherwise mitigates ultraviolet radiation.

According to aspects of the disclosure, the cosmetic formulation comprises cosmetic powder at from about 0.5 wt % to about 80 wt %. According to aspects of the disclosure, the cosmetic powder is selected from the group consisting of metal oxides, silicates, surface modified silicates, organic polymers, and mixtures thereof.

According to aspects of the disclosure, silicates may include hydrated silicates and may include materials, such as but not limited to: silica, mica, talc, sericite, kaolin, and mixtures thereof.

According to aspects of the disclosure, at least a portion of the cosmetic powder is a “substrate” of the covalent, surface-modifying reactions of the present disclosure. According to aspects of the disclosure, at least a portion of the cosmetic powder is a “filler” wherein the covalent, surface-modifying reactions of the present disclosure are not applied.

According to aspects of the disclosure, the silicates may be modified by having organic materials bonded onto a surface thereof. Non-limiting examples of surface-modified silicates include:
silica coated with triethoxycaprylylsilane, aluminum myristate, and disodium stearoyl glutamate;
talc coated with triethoxycaprylylsilane, aluminum myristate, and disodium stearoyl glutamate;
kaolin coated with triethoxycaprylylsilane, aluminum myristate, and disodium stearoyl glutamate;
aluminum calcium sodium silicate coated with triethoxycaprylylsilane, aluminum myristate, and disodium stearoyl glutamate;
mica coated with triethoxycaprylylsilane, aluminum myristate, and disodium stearoyl glutamate;
titanated micas such as Flamenco Velvet, which is a pearl pigment, i.e., a mica coated with triethoxycaprylylsilane, aluminum myristate, disodium stearoyl glutamate, and titanium dioxide.

According to an aspect of the disclosure, at least one hydroxyl of a cosmetic powder substrate may be covalently linked through a polyvalent metal to at least one organic compound comprising at least an organic acid (e.g., fatty acid) or acyl moiety (e.g., acyl amino acid). According to an aspect of the disclosure, the at least one organic compound may bind or absorb at least a portion of an organic UV-active species (e.g., octinoxate, oxybenzone or avobenzone).

According to an aspect of the disclosure, a cosmetic powder substrate may be bound to a surface-treatment agent comprising a complex of triethoxycaprylylsilane, aluminum dimyristate, and disodium stearoyl glutamate. According to aspects of the disclosure, the cosmetic formulation may further comprise at least one inorganic UV-active material. According to an aspect of the disclosure, the triethoxycaprylylsilane complex may bind or absorb at least a portion of an organic UV-active species.

According to aspects of the disclosure, non-limiting inorganic UV-active materials include titanium dioxide and zinc oxide. As used herein, the term “inorganic UV-active material” refers to a particulate, such as particulate titanium dioxide or zinc oxide. As is understood by persons of skill in the art, the titanium dioxide coating of a titanated mica is not UV-active.

According to aspects of the disclosure, the cosmetic formulation may optionally comprise a color pigment. Non-limiting, optional color pigments may include an iron oxide, such as a red, yellow, or black iron oxide. The color pigment may be a submicron particle, preferably from about 0.2 to about 0.3 micron. Typically, cosmetic formulations containing color pigments are foundation formulations, including liquid foundations, pressed powder foundations, and powder foundations.

According to certain aspects, the disclosed formulations may contain an emulsifying agent. According to aspects, the emulsifying agent may be particularly suitable for stabilizing mineral substances.

According to aspects of the disclosure, the cosmetic formulation may further comprise cosmetically-approved emollients (oil, waxes, etc.), humectants (polyols such as glycerin, butylene glycol and pentylene glycol), polysaccharides, and amino acids, preservatives, fragrances, and other additives typically used in cosmetic formulations.

According to aspects of the disclosure, the cosmetic formulations are substantially visually transparent.

The extent to which color bulk powders and dispersions are extended can be ascertained, for example, by determining a change or shift in color, shade, hue, chroma (saturation) or lightness upon mixing or blending. In a particular non-limiting example, a change in color is determined by the formula [(Lₙ₊₁−Lₙ)²+(aₙ₊₁−aₙ)²+(bₙ₊₁−bₙ)²]^{1/2}, which provides a ΔE value. A fully extended color bulk powder has ΔE value of less than about one, as determined by the formula [(Lₙ₊₁−Lₙ)²+(aₙ₊₁−aₙ)²+(bₙ₊₁−bₙ)²]^{1/2}, after the powder is blended. A partially extended color bulk powder has a ΔE value of less than about 2.0, as determined by the formula [(Lₙ₊₁−Lₙ)²+(aₙ₊₁−aₙ)²+(bₙ₊₁−bₙ)₂]^{1/2}, after the powder is blended.

Still other aspects and advantages of the present embodiments will become readily apparent by those skilled in the art from the following detailed description, wherein it is shown and described preferred embodiments, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF THE DRAWINGS

FIG. 1 is a schematic diagram of a substrate with different colored pigments randomly distributed on the substrate surface.

FIGS. 2A-2C are schematic diagrams of A) a single substrate of fully extended color bulk powder according to some embodiments; B) a plurality of substrates of fully extended color bulk powder according to some embodiments with different colored pigments randomly distributed on the substrate surface; and C) a photomicrograph of a plurality of substrates of fully extended color bulk powder with different colored pigments distributed on substrate surface (600× magnification).

FIGS. 3A-3C are schematic diagrams of A) a single substrate of conventional color technology; B) a plurality of substrates of conventional color technology with colored pigments, including aggregates or agglomerates, that surround or are distributed unevenly, irregularly and/or non-uniformly on substrate; and C) a photomicrograph of conventional color technology substrates (480× magnification).

FIGS. 4A and 4B show comparison flowcharts of A) producing fully extended color bulk powders and dispersions; and B) conventional manufacturing process.

FIG. 5 is a schematic representation of the reaction of a mono-carboxylate, surface-treatment agent with a cosmetic powder substrate.

FIG. 6 is a schematic representation of the reaction of a di-carboxylate, surface-treatment agent with a cosmetic powder substrate.

### DETAILED DESCRIPTION

Co-pending U.S. patent application Ser. No. 11/142,468, assigned to the assignee of the present application, discloses a coated powder material containing a powder material having a surface layer that has been chemically immobilized with one or more surface-active agents and coated with an oil. Co-pending application Ser. No. 11/142,468 further discloses methods of making the coated powders. The present application incorporates by reference the entire content of application Ser. No. 11/142,468. The disclosed coated powder materials are dustless powders suitable for incorporation in various cosmetic and toiletry products.

Co-pending U.S. patent application Ser. No. 12/273,495, assigned to the assignee of the present application, discloses a water based slurry compositions, and methods for preparing water based slurry compositions. The disclosed water based slurry composition includes one or more pigments and a substrate, wherein the pigment or substrate has a surface that has been chemically immobilized with at least one surface-treatment agent (e.g., hydrophobic or hydrophilic); wherein the pigment adheres to the substrate, and wherein the pigment and substrate are dispersed in a water medium. The disclosed water based slurry composition also includes one or more pigments and a substrate, wherein the pigment or substrate has a surface that has been chemically immobilized with at least two surface-treatment agents (e.g., hydrophobic or hydrophilic); wherein the pigment adheres to the substrate, and wherein the pigment and substrate are dispersed in a water medium. A method for preparing a water based slurry composition includes providing at least one pigment and a substrate; contacting the substrate or pigment with a surface-treatment agent to produce a surface-modified substrate or pigment material, thereby producing a substrate having adhered thereto the pigment; blending the material until it is fully or partially extended, and dispersing the blended material in a liquid water based (aqueous) medium. The disclosed compositions are suitable for cosmetic applications. The present application incorporates by reference the entire content of application Ser. No. 12/273,495.

U.S. Pat. No. 6,482,441, assigned to Miyoshi Kasei, Inc., discloses surface-treated powders, suitable for cosmetic purposes. The present application incorporates by reference the entire content of U.S. Pat. No. 6,482,441. U.S. Pat. No. 6,482,441 discloses that powder materials may have various functional properties, such as: adhesion, aesthetic feel (touch), covering power, coloring power, and optical absorption and scattering. The '441 patent further discloses such powder properties become more fully realized as the particles are dispersed as primary-sized particles; these properties are attenuated as the particles become flocculated or agglomerated.

U.S. Pat. No. 6,036,945, and a continuation thereof (U.S. Pat. No. 6,280,710) assigned to Shamrock Technologies, Inc., discloses the use of micron-sized particles to nucleate the crystallization of a wax which may contain a sunscreen active. Titanium dioxide and zinc oxide are disclosed as preferred nucleation agents. The '945 patent discloses that because titanium dioxide and zinc oxide are UV-active they additively enhance the SPF value of the resulting wax powders.

According to some embodiments, there is provided fully and partially extended color bulk powders and partially and fully extended color bulk dispersions. In some embodiments, the fully and partially extended color powders and fully and partially extended color dispersions can be used in cosmetic and makeup products, personal care products, and pharmaceutical products. Fully and partially extended color bulk powders and fully and partially extended color bulk dispersion can therefore be referred to as a cosmetic, makeup, personal care or pharmaceutical precursor, since they are typically included in, further modified or added to produce a finished cosmetic, makeup, personal care or pharmaceutical product. For example, a fully and partially extended color powder of a single shade (e.g., fair shade) can be included in a cosmetic or makeup, personal care, or pharmaceutical product. Fully and partially extended color powders of different shades (e.g., light pink, dark yellow, yellowish red, dark brown, etc., shades) can be blended and then subsequently included in a cosmetic or makeup, personal care, or pharmaceutical product.

The term “fully extended,” when used in reference to a color bulk powder or color bulk dispersion, means that little or no change or shift in color, shade, hue, chroma (saturation) or lightness occurs when blending the powder or dispersion, for example, through an atomizer or pulverizer. Thus, because the color bulk powder or color bulk dispersion exhibits little or no change or shift in color, shade, hue, chroma (saturation) or lightness when blending, no additional blending or adjustment of color, shade, hue, chroma (saturation) or lightness in a fully extended color bulk powder or color bulk dispersion is required before formulation or inclusion in a cosmetic, makeup, personal care or pharmaceutical product because color, shade, hue, chroma (saturation) or lightness of the bulk powder or bulk dispersion is fully extended.

Fully extended color bulk powders and fully extended color bulk dispersions provide various superior properties over existing cosmetic, makeup, and personal care products. For example, fully extended color bulk powders and fully extended color bulk dispersions have a relatively stable color, shade, hue, chroma (saturation) or lightness, and can resist a change or shift in color, shade, hue, chroma (saturation) or lightness before and after applying to skin; streaking; shade changes on skin over time; uneven or irregular spreadability that can lead to uneven wear of the cosmetic or makeup; and easy breakage of pressed cakes. Thus, fully extended color bulk powders and fully extended color bulk dispersions can maintain color, shade, hue, chroma (saturation) or lightness consistency when applied to the skin—the appearance of color, shade, hue, chroma (saturation) or lightness prior to applying to skin typically does not substantially change after initially applying to the skin, or after a period of time after applying to the skin (e.g., 1, 2, 3, 4-6, 6-12, or more hours). Additional non-limiting examples of such properties include: a more even distribution when applied to the skin and a more stable color, shade, hue, chroma (saturation) or lightness that is less susceptible to loss or streaking. Further non-limiting examples of such properties include: improved spreadability, which leads to less creasing after applying to skin.

Fully extended color bulk powders and fully extended color bulk dispersions are characterized by little if any change in one or more of color, shade, hue, chroma (saturation) or lightness upon blending, mixing, pulverizing or atomizing the powder or dispersion. In contrast, a color bulk powder or a color bulk dispersion that is not fully extended can be characterized as undergoing a detectable change in one or more of color, shade, hue, chroma (saturation) or lightness upon blending, mixing, pulverizing or atomizing the color bulk powder or dispersion. A color bulk powder or a color bulk dispersion that is partially extended is also characterized as exhibiting a detectable change or shift in one or more of color, shade, hue, chroma (saturation) or lightness upon blending, mixing, pulverizing or atomizing the color bulk powder or dispersion, but the change or shift that occurs in one or more of these parameters is less than that of a not fully extended color bulk powder or color bulk dispersion. For example, as discussed herein, the degree of color bulk powder or color bulk dispersion extension can be evaluated by measuring a change in color, shade, hue, chroma (saturation) or lightness after blending, mixing, pulverizing or atomizing a color bulk powder or a color bulk dispersion. A partially extended color bulk powder or color bulk dispersion will undergo less of a change in color, shade, hue, chroma (saturation) or lightness after blending, mixing, pulverizing or atomizing than a not fully extended color bulk powder or color bulk dispersion.

Various methods can be used to determine whether a color bulk powder or a color bulk dispersion is fully extended, partially extended or not extended. For example, the degree of extension of a color bulk powder or a color bulk dispersion can be evaluated by measuring color, shade, hue, tint, saturation or luminance before and after blending, mixing, pulverizing or atomizing the powder or dispersion. As a non-limiting example, color of a powder can be initially measured and values assigned to L*, a* and b*, in L-a-b color coordinate system, denoted Lₒ*, aₒ* and bₒ*. L* represents lightness, with a range of 0-100; a* represents the green to red spectrum, wherein −a* is green and +a* is red; and b* represents the yellow to blue spectrum, wherein −b* is blue and +b* is yellow (see, Example 2). The powder is then blended as described and color measured again to obtain L*, a* and b* values, denoted L₁*, a₁* and b₁*.

Repeating the process of blending and measuring color, to obtain L*, a* and b*values, which are assigned and the minimum number of passes (blending steps) required to obtain a color powder that exhibits little or no color change is calculated as ΔEn, which is [(Lₙ₊₁−Lₙ)²+(aₙ₊₁−aₙ)₂+(bₙ₊₁−bₙ)²]^{1/2}. A ΔE value of less than about one (ΔE<1.0) means that the color is fully extended, or that little or no difference in color before and after the powder is blended. A ΔE value between 1.0 and 2.0 (ΔE=1.0-2.0) means that the color is partially extended. Typically, a partially extended color bulk powder or dispersion will have a ΔE value of less than about one and one-half (ΔE<1.5). A ΔE value greater than 2.0 (ΔE>2.0) means that the color is not extended.

Typically, a color bulk powder or dispersion that is not fully extended lacks development of one or more of color, shade, hue, chroma (saturation) or lightness as compared to a fully extended color powder or dispersion. A not fully extended color powder or dispersion therefore requires additional components (e.g., pigments, substrates, oils, etc.) or processing steps (e.g., dispersing pigments or substrates by an atomizer, pulverizer, or three-roll-mill) in order to provide a stable color, shade, hue, chroma (saturation) or lightness that is not typically required or is optional for a fully extended color bulk powder or fully extended color bulk dispersion.

Extended color bulk powder or extended color bulk dispersion can be blended together in order to produce a different color, shade, hue, chroma (saturation) or lightness. For example, to adjust or change the color, shade, hue, chroma (saturation) or lightness of a fully or partially extended color powder or dispersion, a particular color, shade, hue, chroma (saturation) or lightness can be mixed with one or more other powder(s) having a different color, shade, hue, chroma (saturation) or lightness by blending together in a mixer. If two or more fully extended color bulk powders or fully extended color dispersions are to be mixed with each other, they may be blended in a mixer to produce a desired color, shade, hue, chroma (saturation) or lightness—there is no need to blend the mixture through an atomizer, pulverizer or three roll mill. Thus, fully extended color bulk powders and color bulk dispersions can be adjusted for color, shade, hue, chroma (saturation) or lightness by simple blending without requiring multistep processing (e.g., atomizing, pulverizing), and are therefore more readily formulated or included in final cosmetic, makeup, personal care and pharmaceutical products.

Fully and partially extended color bulk powders and color bulk dispersions comprise powder materials, which include substrates and pigments acceptable for formulation or inclusion in a final cosmetic, makeup, personal care or pharmaceutical product. Thus, a fully or partially extended color bulk powder or fully or partially extended color bulk dispersion can have one or more, or all substrates or pigment or other cosmetically acceptable components suitable for formulation or inclusion in a final cosmetic, makeup, personal care or pharmaceutical product.

Substrates and pigments typically comprise or consist of a material compatible or acceptable for cosmetic and makeup products, personal care products and pharmaceutical products. Substrates and pigments are typically in the form of a powder, which is a solid, dry material consisting of extremely small, flowable particles. Particular classes of powder materials are inorganic and organic particles, beads, crystals, clays, metals, metal oxide powders, or plastics suitable for cosmetic use.

A dispersion is a mixture in which a material is distributed or diffused in a medium, such as a liquid, solid or gas. A liquid is a smooth, amorphous substance in the fluid state of matter having no particular fixed shape (free flowing) and relatively invariable volume. Dispersions include an oil liquid, oil in water phase (o/w) and water in oil phase (w/o). Oil in water phase (o/w), water in oil phase (w/o) and water in silicone dispersions are typically referred to as emulsions.

Fully and partially extended color bulk powders and dispersions include at least one substrate and one pigment. Fully and partially extended color bulk powders and dispersions also include a plurality or mixture of different substrates, a plurality or mixture of different pigments, or a plurality or mixture of substrates and pigments.

Typical substrate sizes are about 1-30 microns in diameter, usually not less than 1 micron, for example, have a primary size of about 1-3 microns. Substrate particles are typically larger than pigment particles and have various shapes, for example, spherical, elliptical or “platy.” Substrates provide desirable texture and other characteristics such as smoothness, silkiness, round feel, moisture feel, optical benefits (soft focusing, hiding or concealing wrinkles or blemishes), etc.

Specific non-limiting examples of substrates include clay, mica (e.g., pearl colored mica, such as Timron Super Silver™, a mica coated with titanium dioxide produced by Rona/EMD Industries), talc, kaolin, sericite, silica (e.g., silica beads such as aluminum silicate, magnesium silicate and calcium sodium silicate, beadyl beads™, fumed silica), alumino-silicate minerals (zeolites), nylon (e.g., nylon beads or nylon powder), acrylates such as polymethyl methacrylate (PMMA or powder), metal powders (such as aluminum), ceramic powders (such as silicon nitride or boron nitride), cotton powder, wool powder, silk powder, cellulose and cellulose powder, urethane, styrene, polystyrene and polystyrene powder, polyolefin, polyethylene and polyethylene powder, polyamide, zirconium, aluminum oxide, zirconium oxide, starch, starch powder and starch derivatives such as aluminum starch octenylsuccinate, and calcium carbonate (chalk).

Substrates include “extenders.” An extender can function as a filler or bulking agent for powders and dispersions as set forth herein or known to the skilled artisan (e.g., pressed foundation, loose powder, blush, consealer, etc.). Extenders as a class typically have a size, shape or structure that is similar or identical to substrates as disclosed herein and understood by the skilled artisan. The term extender is typically used to refer to a substrate material that is added to a color bulk powder or color bulk dispersion after surface treatment or modification of pigment or substrate.

Extenders include natural and synthetic substrates that may or may not have a color, shade, hue, chroma (saturation) or lightness that may vary in saturation and luminance. As with a substrate, an extender has a size typically greater than 1 micron (1 μm), for example about 1-30 microns, and can have various shapes, for example, spherical, elliptical or “platy.”

Non-limiting examples of extenders include talc, kaolin (clay), natural and synthetic micas including muscovite mica and sericite, titanated mica, cotton powder, starch, magnesium carbonate, calcium carbonate, aluminum silicate, magnesium silicate, calcium silicate, synthetic silicates, clay, bentonite, montmorillionite, calcite, chalk, bismuth oxychloride, boron nitride, fumed silica, silica beads, plastic beads such as acrylics, nylons such as Nylon 12, nylon beads, aluminum, calcium, or sodium silicate, and barium sulfate.

Amounts of substrate in fully and partially extended color bulk powders and color bulk dispersions, intermediates thereof and preparation methods may vary depending upon the precursor or intermediate product to be produced, the ultimate or final cosmetic, makeup, personal care or other product to be produced, or method of manufacture. In a fully or partially extended color bulk powder, weight percent of a substrate is typically about 0 to 95%. In a fully or partially extended color bulk dispersion, weight percent of a substrate is typically about 0 to 95%.

Although not wishing to be bound by theory, many pigment particles typically adhere to a substrate particle when pigment size is smaller than substrate size. In situations where pigment size is larger than substrate size, for example pearl pigments can be from about 50-100 microns in size with a platy structure, many substrate particles can adhere to a pigment particle. The term “adhere” used herein refers to either situation. Thus, the terminology “pigment adheres to the substrate” also includes “substrate adheres to the pigment.”

Pigment adhered to substrate and substrate adhered to pigment show skin shades. Pigments adhered onto substrates or substrates adhered onto pigments may be uniformly (evenly) or non-uniformly (unevenly) distributed.

As used herein, the term “pigment,” which includes “dyes” is natural or synthetic material that has a certain color, shade, hue, chroma (saturation) or lightness. Pigments may be organic or inorganic in chemical nature. Pigments typically have a primary particle diameter not greater than about 3 microns. Pigments more typically are about one order of magnitude smaller in size than substrates, for example, about 0.1-1.0 microns in diameter. Other pigments, such as pearl pigments typically have a larger size, for example 10, 20, 30, 40, or 50-100 microns (μm).

Non-limiting examples of inorganic pigments include white titanium dioxide pigments (e.g., rutile, anatase, and ultrafine TiO₂), zinc oxides (e.g., ultrafine ZnO), which can be of pigment grade and have a primary size of about 0.3 μm, or ultrafine grade, and have a primary size of less than about 0.1 μm. Other inorganic pigments include zirconium oxide, zirconium dioxides, iron oxides (including yellow, red, brown, green and black iron oxides), ultramarines (such as ultramarine blue, ultramarine violet, ultramarine pink, etc.), pearl pigments (e.g., mica, titanated mica, bismuth oxychloride, etc.), manganese violet, Prussian blue, chromium oxides, chromium hydroxides, and carbon black. Non-limiting examples of organic pigments include “lake” dyes, β-carotene, carmine, chlorophyll and the like.

Fully and partially extended color bulk powders and color bulk dispersions typically include one or more different pigments. A plurality of different pigments (e.g., 2, 3, 4, 5, 6, 7, 8, 9, 10 or more pigments that optionally have a different color, shade, hue, chroma (saturation) or lightness) can be included to produce a “composite” of pigments. A plurality of different pigments, optionally having a different color, shade, hue, chroma (saturation) or lightness, can therefore be included in fully and partially extended color bulk powders and color bulk dispersions. Such extended color bulk powders and extended color bulk dispersions can be conveniently referred to as “composite” extended color bulk powders and extended color bulk dispersions.

Amounts of pigments and dyes to employ in fully and partially extended color bulk powders and color bulk dispersions, intermediates thereof and production methods may vary depending upon the desired color, shade, hue, chroma (saturation) or lightness. As set forth in Example 2, pigment types, absolute amounts and relative ratios span a broad range and can be selected based upon a desired color, shade, hue, chroma (saturation) or lightness, physical (e.g., size, shape), functional or chemical characteristic or property. In a fully or partially extended color bulk powder, total weight percent of pigments is typically about 3 to 20%, 5 to 20%, 5 to 15%, 5 to 18%, 8 to 12%, or 10 to 25%. In a fully or partially extended color bulk dispersion, total weight percent of a substrate is typically about 3 to 20%, 5 to 20%, 5 to 15%, 5 to 18%, 8 to 12%, or 10 to 25%.

Ratios of pigments and substrates can also vary depending upon the precursor product to be produced, the ultimate cosmetic, makeup, personal care or other product to be produced, or method of manufacture. Exemplary pigment to substrate ratio in a cosmetic or makeup product is from about 5:95 to 95:5. In personal care and other products, the ratio is typically not limited.

Substrates and pigments can be deflocculated or deagglomerated. According to some embodiments, there are fully and partially extended color bulk powders and color bulk dispersions, intermediates thereof and production methods that include or employ deflocculated and/or deagglomerated substrates, pigments and other cosmetically suitable materials. Substrates, and pigments can be deflocculated or deagglomerated by any physical or chemical means which achieves at least some degree of dispersal of aggregates or agglomerates. Non-limiting examples of physical deagglomerating include shearing and grinding.

As used herein, the term “surface-treatment agent” refers to chemical agents that have the ability to modify, alter or react with the surface of a powder material by forming chemical bonds on the surface of the powder. Specific non-limiting classes of surface treatment agents include surface active agents, which include surfactants, detergents, wetting agents and emulsifiers. Surface-active agents may be nonionic, anionic, cationic, amphoterics, hydrophobic or hydrophilic.

Surface-treatment agents typically have one or more reactive groups, such as a hydrophilic moiety (e.g., a carboxyl group, a phosphorous group, a sulfur group, a silanol group or a silane group) or hydrophobic moiety (e.g., a hydrocarbon, a dialkyl(CH₃—, C₂H₅—) polysiloxane, perfluoroalkyl, etc.) in their structure. Surface-treatment agents may or may not contain one or more hydroxyl groups or alkylene oxide moieties, such as ethylene oxide or propylene oxide. Those having hydroxy groups in their structure and hydrophilic characteristics can be delivered after completing the reaction onto the surface.

Non-limiting examples of surface treatment agents include acyl collagens, ether carboxylic acids, lactic acid, gluconic acid, galacturonic acid, glucarolactone, gallic acid, glucoheptanoic acid, amino acids (such as thereonine and serine) and their salts, acyl amino acids (such as acylglutamates, acylsarcosinates, acylglycinates, and acylalaninates), fatty acids and their salts, and glycerol phosphate esters (such as lecithin). Additional non-limiting examples of surface-treatment agents include methicone, dimethicone and polyethylenes with free carboxylic acids.

Examples of anionic surface active agents (surfactants) include soaps (fatty acids/alkyl carboxylic acids salt), hydroxy fatty acids, alkyl sulfate, alkyl ether phosphate, polyoxyalkylene alkyl ether sulfate, polyoxyalkylene alkyl ether carboxylate, alkylether phosphate, acyl N-methyl taurate, N-acylamino acid salts (including glutamate, sarcosinate, lalaninate, glycinate, B-alaninate), acyl peptides (acyl collagen, acyl silk protein), sodium cocoate, stearic acid, iso-stearic acid, potassium palmitate, sodium laurate, 12-hydroxystearic acid, sodium lauryl sulfate, sodium myristyl phosphate, sodium myristoyl sarcosinate, sodium polyoxyethylene lauryl sulfate, polyoxyethylene myristyl carboxylate, potassium myristate, zinc gluconate, isostearyl sebacic acid, aluminum myristate, sodium myristoyl taurate, disodium stearoyl glutamate, disodium cocoyl glutamate, arginine lauryl glycinate, sodium dilauramidoglutamide lysine.

Exemplary fatty acid surface treatment agents include structures and salts of [Formula I]:

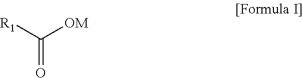

wherein R₁ is selected from the group consisting of alkyl, alkylamide, alkenyl, alkynyl, alkoxy, aryl, cycloalkyl, and arylalkyl group, all of which may be substituted by one or more hydroxy group, and may further be substituted by one or more alkoxyl, carboxyl, or oxo group. R₁ has a carbon number of C₈ to about C₂₄; and M is hydrogen, or metal or its equivalent (organic base such as triethanolamine, aminomethyl propanol, lysine, etc.).

Exemplary amphoteric surfactant surface treatment agents include laurylamidobetaine, stearyl amphoacetate, lauryl amphopropionate, stearyl amphopropionate, alkyl dimethylaminoacetic acid beanie, alkyl amidopropyldimethyl aminoacetic acid betaine and alkyl-N-carboxymethyl-N-hydroxyethylimidazolinium betaine. Additional examples include silane and functionalized silanes (“silanes” to “silanols” by hydrolysis): triethoxyoctylsilane, trichlorobutylsilane, trimethoxy dimethylpolysiloxyl silane. Further non-limiting examples are also described in U.S. Patent Application Publication Nos. 2006/0225616 and 2006/0286048.

Exemplary alkyl ether carboxylic acid surface treatment agents include structures and salts of [Formula II]:

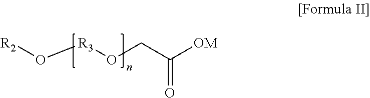

wherein R₂ is selected from the group consisting of alkyl, alkylamide, alkenyl, alkynyl, alkoxy, aryl, cycloalkyl, and arylalkyl group, all of which may be substituted by one or more hydroxy group, and may further be substituted by one or more alkoxyl, carboxyl, or oxo group. R₂ has a carbon number of C₈ to about C₂₄; R₃ includes ethylene, propylene, or butylene; n is 0 to 20; and M is hydrogen, or metal or its equivalent (organic base such as triethanolamine, aminomethyl propanol, lysine, etc.).

Exemplary acylamino acid surface treatment agents include structures and salts of [Formula III]:

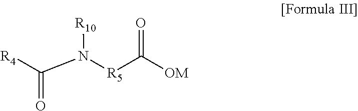

wherein R₄ and R₅ are each independently selected from the group consisting of alkyl, alkylamide, alkenyl, alkynyl, alkoxy, aryl, cycloalkyl, and arylalkyl, each of which may be substituted by one or more hydroxy group, and may further be substituted by one or more alkoxyl, carboxyl, or oxo group; R₄ has a carbon number of C₈ to about C₂₄; R₁₀ is hydrogen or methyl; and M is hydrogen, or metal or its equivalent (organic base such as triethanolamine, aminomethyl propanol, lysine, etc.).

Additional surface treatment agents include compounds having a structure represented by [Formula IV]:

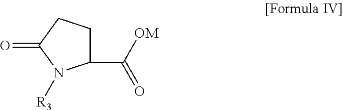

wherein R₃ is selected from the group consisting of alkyl, alkylamide, alkenyl, alkynyl, alkoxy, aryl, cycloalkyl, and arylalkyl, all of which may be substituted by one or more hydroxy group, and may further be substituted by one or more alkoxyl, carboxyl, or oxo group. R₃=C₈ to C₂₄; M is hydrogen, or metal or its equivalent (organic base such as triethanolamine, aminomethyl propanol, lysine, etc.).

Further surface treatment agents include compounds having structure represented by [Formula V]:

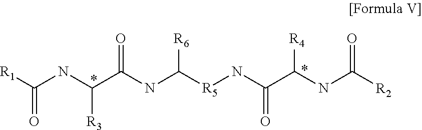

wherein R₁ and R₂ are each independently selected from an alkyl, alkylamide, alkenyl, alkynyl, alkoxy, aryl, cycloalkyl, or arylalkyl, each of which may be substituted by one or more hydroxy group, and may further be substituted by one or more alkoxyl, carboxyl, or oxo group. hydrophobic moieties, R₁, R₂ are each independently C₈ to C₂₄; R₃ and R₄ are each independently is selected from the group consisting of alkyl, alkynyl, alkenyl, and amino acid residual moieties; R₅ and R₆ are each independently is selected from the group consisting of alkyl, alkynyl, and alkenyl; at least one of R₃, R₄ and R₆ may be substituted with a carboxylic group, which is either in its acid form or a salt form (metal or its equivalent, e.g., organic base such as triethanolamine, aminomethyl propanol, lysine, etc.).

Exemplary phospholipid and alkyl ester phosphoric acid surface treatment agents include structures and salts represented by [Formula VI]:

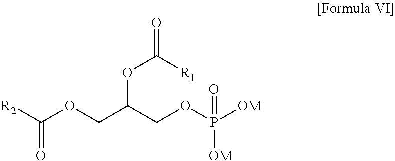

wherein R₁ and R₂ are each independently selected from the group consisting of alkyl, alkylamide, alkenyl, alkynyl, alkoxy, aryl, cycloalkyl, and arylalkyl, each of which may be substituted by one or more hydroxy group, and may further be substituted by one or more alkoxyl, carboxyl, or oxo group; R₁ and R₂ each independently have a carbon number of C₈ to about C₂₄; and M is hydrogen, or metal or its equivalent (organic base such as triethanolamine, aminomethyl propanol, lysine, etc.).

Particular embodiments of Formula VI include diester metallates and monoester dimetallates as shown below:

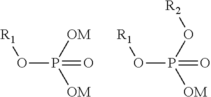

wherein R₁, R₂, and M are as defined above.

Exemplary amphoteric surface treatment agents include structures represented by [Formula VII]:

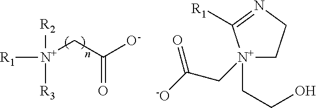

wherein R₁, R₂ and R₃ are each independently selected from the group consisting of alkyl, alkylamide, alkenyl, alkynyl, alkoxy, aryl, cycloalkyl, and arylalkyl, each of which may be substituted by one or more hydroxy group, and may further be substituted by one or more alkoxy, carboxyl, or oxo group; and R₁ has a carbon number of C₈ to about C₂₄.

Exemplary silane and silane derivative surface treatment agents include structures represented by [Formula VIII]:

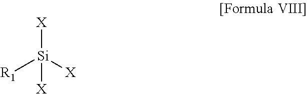

wherein R₁ is selected from the group consisting of alkyl, alkylamide, alkenyl, alkynyl, alkoxy, aryl, cycloalkyl, and arylalkyl, each of which may be substituted by one or more hydroxy group, and may further be substituted by one or more alkoxy, carboxyl, or oxo group; R₁ has a carbon number of C₈ to about C₂₄; and X is alkoxy (e.g., methoxy, ethoxy, isopropoxy, isobutoxy) or halogen (F, Cl, Br, etc.).

Additional embodiments of silane derivatives include:

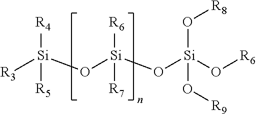

wherein R₃, R₄, R₅, R₆, R₇, R₈ and R₉ are each independently selected from the group consisting of an alkyl, alkylamide, alkenyl, alkynyl, alkoxy, aryl, cycloalkyl, and arylalkyl, each of which may be substituted by one or more hydroxy group, and may further be substituted by one or more alkoxyl, carboxyl, or oxo group; and n is 0 to 60.

Still further embodiments of surface treatment agents include compounds having a structure [Formula IX]:

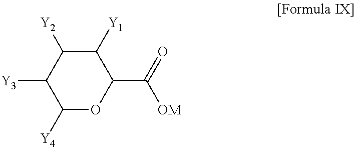

wherein Y₁, Y₂, Y₃, Y₄ are each independently selected from the group consisting of hydrogen, hydroxy, alkoxy, and oxo, wherein at least one of Y₁, Y₂, Y₃, Y₄ is a hydroxy group; and M is hydrogen, or metal or its equivalent (organic base such as triethanolamine, aminomethyl propanol, lysine, etc.).

Other embodiments of surface treatment agents include compounds having a structure [Formula X]:

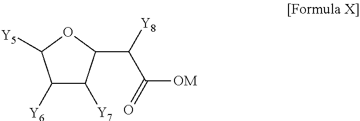

wherein Y₅, Y₆, Y₇, Y₈ are each independently selected from the group consisting of hydrogen, hydroxy, alkoxy, and oxo, wherein and at least one of Y₅, Y₆, Y₇, Y₈ is a hydroxy group; M is hydrogen, or metal or its equivalent (organic base such as triethanolamine, aminomethyl.

Amounts of a surface treatment agent to employ in fully and partially extended color bulk powders and color bulk dispersions, intermediates thereof and production methods may vary depending upon the modification desired, the precursor product to be produced, the ultimate cosmetic, makeup, personal care or other product to be produced, or method of manufacture. For example, a surface-treatment agent may be used in an amount of at least 0.1% by weight, based on the weight of the powder material. Surface-treatment agents are typically present in an amount ranging from about 1.0 to about 200% by weight; or, from about 1.0 to about 60% by weight; or, from about 3.0 to about 30% by weight.

The amount of a surface-treatment agent can depend, at least in part, on the specific surface area of target pigment(s), extender(s) and substrate(s). For example, for regular iron oxide pigments, 2 to about 10 parts by weight of surface-treatment agent per 100 parts of powder. For an ultrafine powder, such as silica having a large surface area, 15 to about 100 parts by weight per 100 parts of powder. Thus, the greater the surface area, the more surface-treatment agent used.

In a particular non-limiting example, for a mixture of one or more pigments with one or more substrates, a surface treatment agent is in an amount of about 0.5 to 400 parts per 100 parts of pigment and substrate. In a particular non-limiting example of a fully extended color bulk powder, the weight percent of a surface treatment agent is typically about 0.5 to 10%. In a particular non-limiting example of a fully extended color bulk dispersion, the weight percent of a surface treatment agent, based upon the total weight of the pigment(s)+substrate(s) is typically about 0.5 to 20%, or about 1.0 to 15%.

One or more pigments and one or more substrates are contacted with a surface-treatment agent, and the pigment or substrate is in turn either modified by the agent or the agent is bound to the surface of the pigment or substrate (e.g., absorbed, chemically linked or immobilized; see, for example, U.S. Pat. No. 5,897,868). As an example, a substrate, pigment, or a plurality of different substrates and pigments (e.g., a mixture of different colored pigments), is contacted with a surface treatment agent which in turn becomes modified by the agent or the agent is bound to surface of the substrate and/or pigment. Surface modification of substrates and/or pigments allow the material(s) present to adhere to each other.

A surface treatment agent can be chemically immobilized or adsorbed onto the surface substrate and/or pigment. Chemical linkage or immobilization of surface-treatment agents to a substrate or pigment differs from adsorption in that surface treated material has a more uniformly chemically bound reaction product. Chemical linkage or immobilization tends to reduce movement and/or rearrangement of any material linked or attached onto the surface of the modified powder material. For example, a pigment that is linked or attached to the surface of a substrate by virtue of a surface treatment agent will have less mobility than a pigment that is attached or linked to the surface of a substrate by virtue of adsorption.

In order to facilitate or enhance immobilization of surface-treatment agents to substrate or pigment, a reaction may be created by a water soluble compound having a lipophilic or hydrophilic moiety being absorbed onto the surface of the substrate or pigment. As a non-limiting example, addition of a water-soluble salt of a polyvalent metal, such as magnesium, calcium, aluminum, titanium, zinc or a zirconium salt (e.g., zirconium sulfate or chloride), or an alkaline salt, such as a sodium, potassium, lithium, ammonium, or an amine salt, can produce a chemical linkage. The reaction provides a surface-treatment agent chemically immobilized onto the surface of the substrate or pigment particle. In contrast, coating a substrate or pigment with a surface-treatment agent involves absorbing the surface-treatment agent onto the surface of the substrate or pigment.

During treatment with a surface treatment agent, surface of one or more substrate(s) or pigment(s) become modified and in turn particles of the substrate or pigment adhere to each other. For example, small pigment particles become attached or linked to larger particles, such as substrate particles. Including a cosmetically acceptable oil (a single oil or mixture of oils) during a treatment in which substrate or pigment surface is hydrophobically modified invites oil at the same time as the particles become attached or linked to each other. Surface treatment agents and oil in combination function as a “glue” to attach or link particles, and other components optionally present, to each other. A mixture of two or more different pigments during such surface treatment results in forming color pigment composites, which are typically randomly and uniformly distributed onto the surface. Thus, oils, emulsifiers, etc., can be present in a mixture with one or more substrates and pigments when contacted with a surface treatment agent.

Following surface modification or coating, a powder material can then be admixed or blended with another (e.g., second) powder material, such as a different pigment, or substrate or extender, or another cosmetically acceptable ingredient such as an oil, emulsifier, binder, etc. The second material may or may not have been treated with a surface treatment agent. Alternatively, two or more materials (e.g., different colored pigments), can be combined or mixed together prior to contact with a surface treatment agent, such as in a aqueous slurry, and then subsequently contacted with a surface treatment agent in order to simultaneously produce two or more surface modified or coated materials. Chemical immobilization of a surface-treatment agent on materials can be facilitated by a water soluble compound having a lipophilic or hydrophilic moiety being absorbed onto the surface of the material, as set forth herein or known to the skilled artisan.

In some embodiments, binders can be included in color bulk powders and color bulk dispersions and employed in methods. For example, a binder, such as an oil (emollient) may but need not be present during surface treatment of a powder material such as pigment(s) or substrate(s). A binder may but need not be added prior to, during or following surface treatment of a pigment or substrate. A fully or partially extended color bulk powder or color bulk dispersion may therefore include a binder such as an oil, if desired. Although not wishing to be bound by theory, oil binders reduce movement of pigment. A binder such as an oil may but need not be added prior to or after making a fully or partially extended color bulk powder or color bulk dispersion.

“Binders” in color cosmetic industry typically refer to compounds that provide adhesive properties to material such as substrates and pigments so they remain together. Binders include oils (emollients), fats, waxes, metal soaps as a solid binder (e.g., zinc stearate, magnesium palmitate, etc.), latex emulsions, styrene, styrene butadiene, polyvinyl acetate (PVA), acrylic, acrylic-styrene, acrylic polyvinyl acetate, poylurethanes, and others acceptable for cosmetic and makeup, personal care and pharmaceutical products.

Amounts of a binders to employ in the fully and partially extended color bulk powders and color bulk dispersions, intermediates thereof and production methods may vary depending upon the precursor or intermediate product to be produced or the final or ultimate cosmetic to be produced. In a fully or partially extended color bulk powder, the weight percent of binders is typically about 0 to 25%. In a fully or partially extended color bulk dispersion, the weight percent of binders is typically about 0 to 25%. Binders may be used in an amount of at least 0 to 25% by weight. Additional oils can be added in order to increase the overall amount of binders to about 0 to 50% or 0 to 35%.

Oils include esters and waxes such as glycerides (e.g., monoglycerides, diglycerides and triglycerides), fatty acid esters, hydroxyl acid esters, dimer acid esters, other naturally derived esters (such as castor oil derivatives and vegetable-based oils, such as vegetable squalane), olive oil, camellia oil, macademia nut oil, castor oil. Wax esters include esters of higher fatty acids and higher fatty alcohols, carnauba wax, candelilla wax, jojoba oil, bees wax, lanolin. Fatty esters include isopropyl myristate, isononyl isononanoate, octyldodecyl myristate, cetyl octanoate, diisostearyl malate, caprylic- and capric triglyceride, isodecyl neopentaoate, isosteraryl neopentanoate, cholesteryl- behenyl-, octyldodecyl-lauroyl glutamate, and octylmethoxycinnamate. Petroleum and synthetic oils include hydrocarbons such as paraffins, isoparafin, petrolatum, ceresin, microcrystalline wax, squalane; silicones and derivatives thereof (such as cyclomethicone, cetyldimethicone, diphenyldimethicone, polysilicone-11, etc.), lipophilic vitamins and their derivatives (tocopherol, tocopherol acetate, tocopherol succinate, retinol, retinoic acid, retinyl parmitate, ascorbyl parmitate, etc.), lipophilic dyes, essential oils, and combinations thereof. Higher fatty acids include lauric acid, myristic acid, palmitic acid, stearic acid, isostearic acid, behenic acid, oleic acid, linoleic acid, linolenic acid, ricinoleic acid, docosa-polyenoic acid, erucic acid. Higher alcohols include behenyl alcohol, cetyl alcool, stearyl alcohol, isostearyl alcohol, octyldodecanol. Oils also include silicones: dimethyl polysiloxane (“dimethicone,” available as DC 200 from Dow Corning), methylphenyl polysiloxane, cyclopentasiloxane, cetyl dimethicone, and PEG/PPG dimethicone. Other oils include mineral oil, isostearyl neopentanoate, caprylic/capric triglyceride, cetyloctanoate, diisostearyl maleate, octylmethoxycinnamate, isododecane, isononyl isononanoate, ethylhexyl methoxycinnamate, behenyl alcohol, and cholesteryl-, behenyl-, octyldodecyl-lauroyl glutamate, which produce good results. Choosing a particular oil depends in part on the product being produced.

Oil can be applied as a liquid. Oils that are not commercially available as liquids, such as ascorbyl palmitate, which is lipophilic vitamin and sold primarily as a solid, can be solubilized in liquid oil before being used as a coating oil. Suitable solubilizing oils include vitamin E acetate, caprylic/capric triglyceride, and others. Once in a liquid form, the oil may then be added to the material using conventional techniques. For example, the oil may be poured into an intake port during missing and mixed until the composition is homogeneous.

Oil, when present, is typically in an amount ranging from about 0.1 to 180% by weight, based on the weight of the substrate/pigment material. Oil, when present, can be in an amount ranging from about 1.0 to about 150% by weight; or, from about 3.0 to about 120% by weight; or, from about 5.0 to about 60% by weight; or, from about 5.0 to about 25 to 30% by weight.

The combined weight percentage of a surface-treatment agent(s) and oil, if oil is present, is typically at least about 4.0% by weight, based on the weight of the material. Typically, a combined weight percentage ranges from about 4.0 to about 1000%; or, from about 4.0 to about 500% by weight; or, from about 5.0 to about 250% by weight or, from about 8.0 to about 125% by weight or, from about 10 to about 75% by weight.

Emulsifiers, surfactants, dispersants, suspending agents, emulsion stabilizers, defoamers, thickeners and other cosmetically acceptable materials and agents can also be employed in the compositions and methods disclosed herein. Non-limiting examples of emulsifiers include cetyl dimethicone copolyol, polygyceryl-4 isosteatrate, glyceryl stearate, PEG-100 stearate, cetyl alcohol, dicetl phosphate, and ceteth-10 phosphate isostearic acid. An additional suitable emulsifying agent is a hydroxyethylacrylate/sodium acryloyldimethyltaurate copolymer formulated with squalene and polysorbate 60. Such a material is traded under the name Simulgel™ NS. Other suitable emulsifying agents include RM2051® (Dow Corning) a dimethicone based thickening and emulsifying polymer (INCI name: Sodium Polyacrylate (and) Dimethicone (and) Cyclopentasiloxane (and) Trideceth-6 (generic name for polyethylene glycol ethers of tridecyl alcohol having an average of six ethylene oxide units) (and) PEG/PPG 18/18 Dimethicone).

Other conventional additives typically employed in cosmetic powder compositions may be employed. Such additives include, but are not limited to one or more preservatives such as methyl paraben, butyl paraben, propyl paraben, phenoxyethanol, benzoic acid, imidazolidinyl urea and other conventional preservatives, antioxidants, emollients, plasticizers, surfactants water proofing additives, botanical extracts and fillers including polyethylene, magnesium carbonate, methylcellulose, mica and the like.

Surfactants typically include nonionic forms. Non-limiting examples of nonionic surfactants include polyoxyalkylene (PEG or/and PPG) type nonionic emulsifiers having structures:

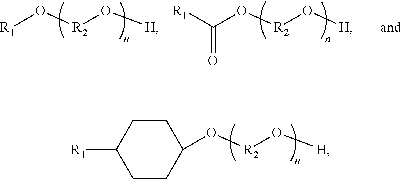

wherein R₁is selected from the group consisting of alkyl, alkylamide, alkenyl, alkynyl, alkoxy, aryl, cycloalkyl, and arylalkyl group, each of which may be substituted by one or more hydroxy group, and may further be substituted by one or more alkoxyl, carboxyl, or oxo group. R₁ has a carbon number of C₈ to about C₂₄; R₂ is selected from the group consisting of —C₂H₄—, —C₃H₆—, and —C₄H₈—.

Further non-limiting examples of nonionic surfactants include polyhydric alcohol ester type emulsifiers wherein at least one of the hydroxy group (—OH) of a “polyol” is esterified with a fatty acid, leaving residual hydroxy groups to function as hydrophilic moieties. Residual hydroxy groups can also be modified by alkylene oxide at different polymerization number. The combination of esterified (hydrophobic) and free hydroxyl (hydrophilic) groups allows the surfactant molecule to act as an emulsifier. Non-limiting examples of polyols having different numbers of hydroxyl groups include glycerine with 3 —OH's; pentaerythritol and sorbitan each with 4 —OH's; sorbitol with 6 —OH's; and sucrose with 8 —OH's. Additional non-limiting examples are shown below:

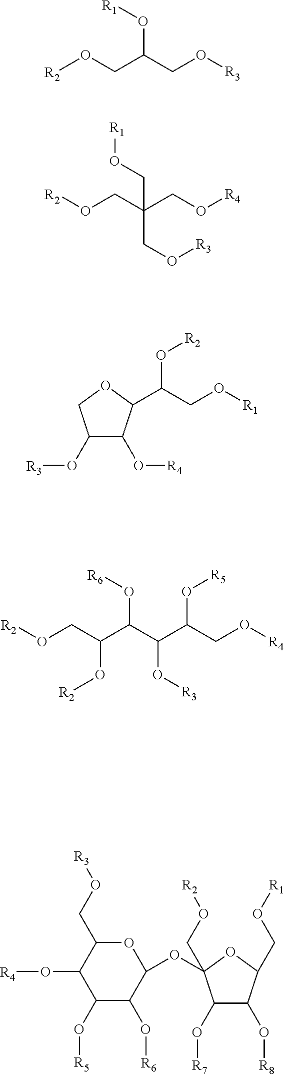

wherein R₁, R₂, R₃, R₄, R₅, R₆, R₇ and R₈ are each independently a moiety of “structure I” or “structure II” and at least one of R₁, R₂, R₃, R₄, R₅, R₆, R₇ and R₈ is “structure I”.

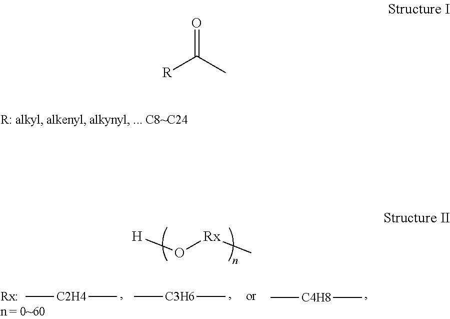

Fully and partially extended color bulk powders and fully and partially extended color bulk dispersions and intermediate products thereof can be produced as set forth herein. According to some embodiments, methods are provided for producing (production, manufacture, preparing) fully and partially extended color bulk powders and fully and partially extended color bulk dispersions, and methods of producing (production, manufacture, preparing) intermediate products of fully and partially extended color bulk powders and fully and partially extended color bulk dispersions.

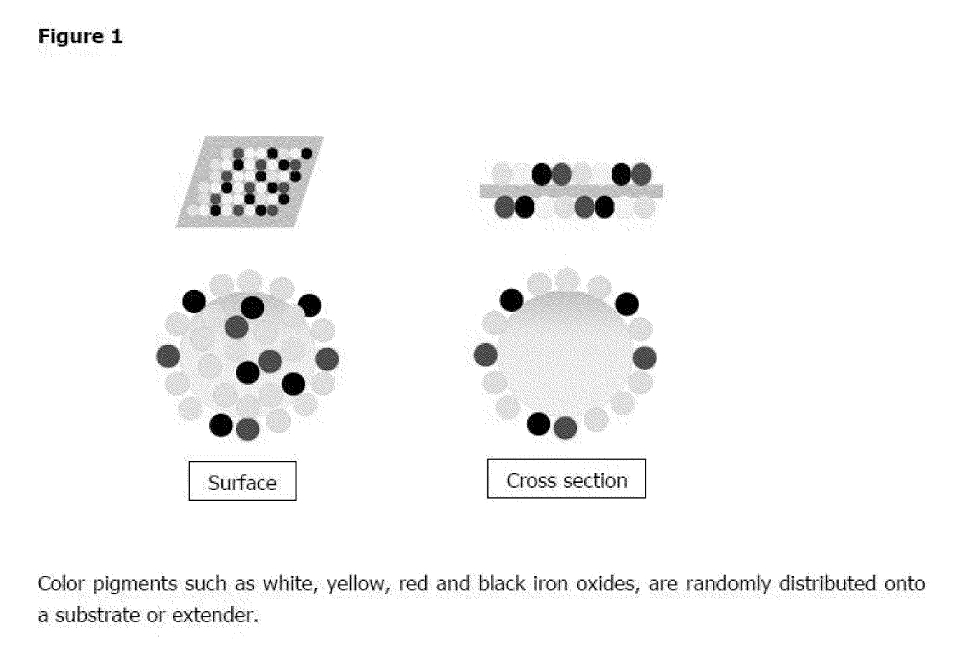

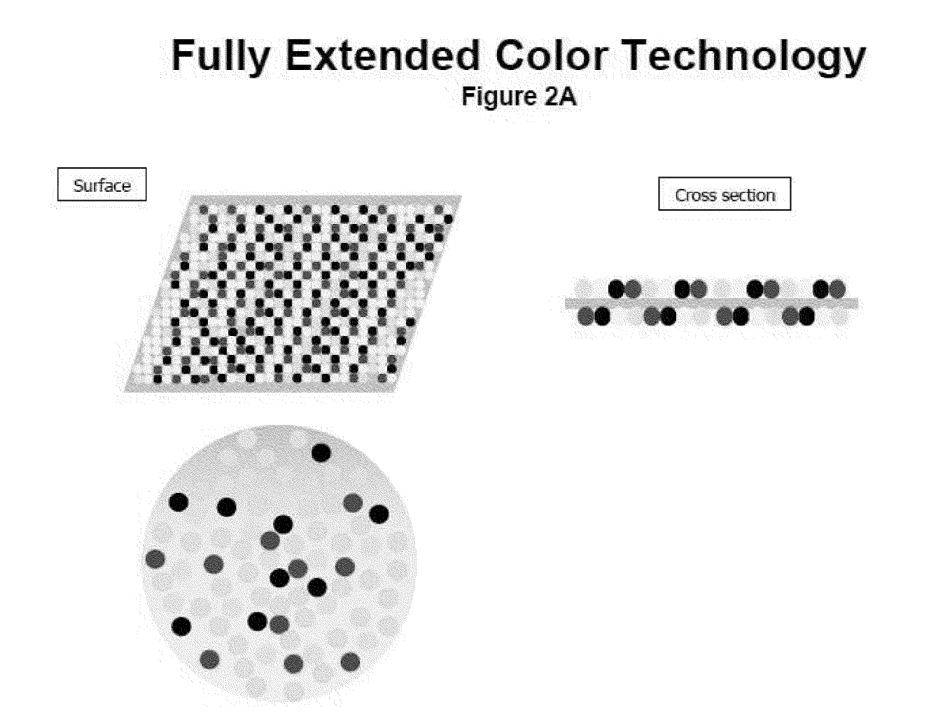

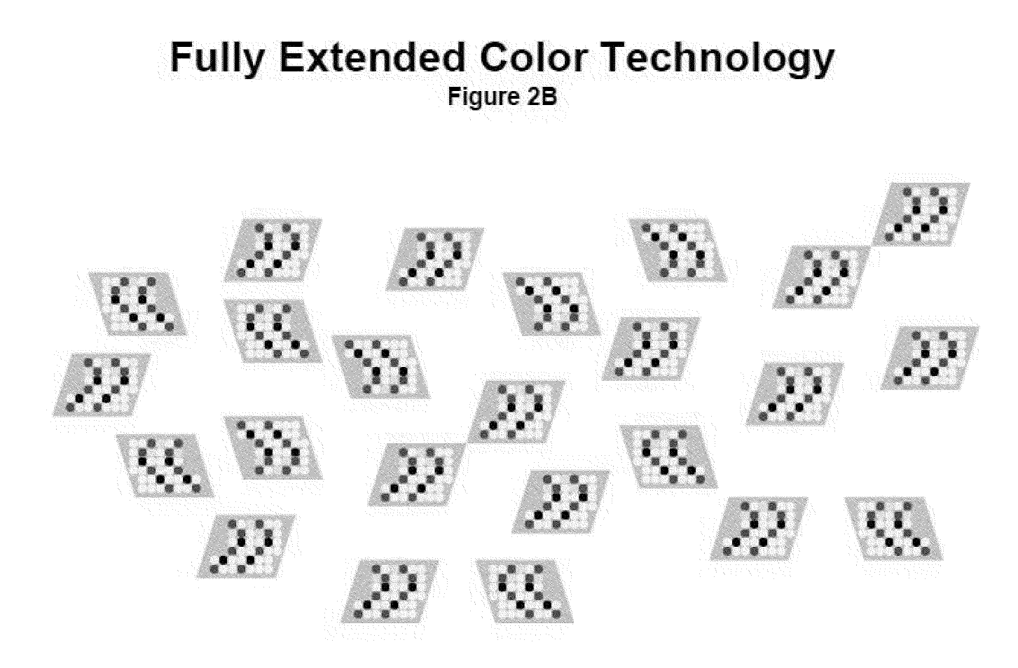

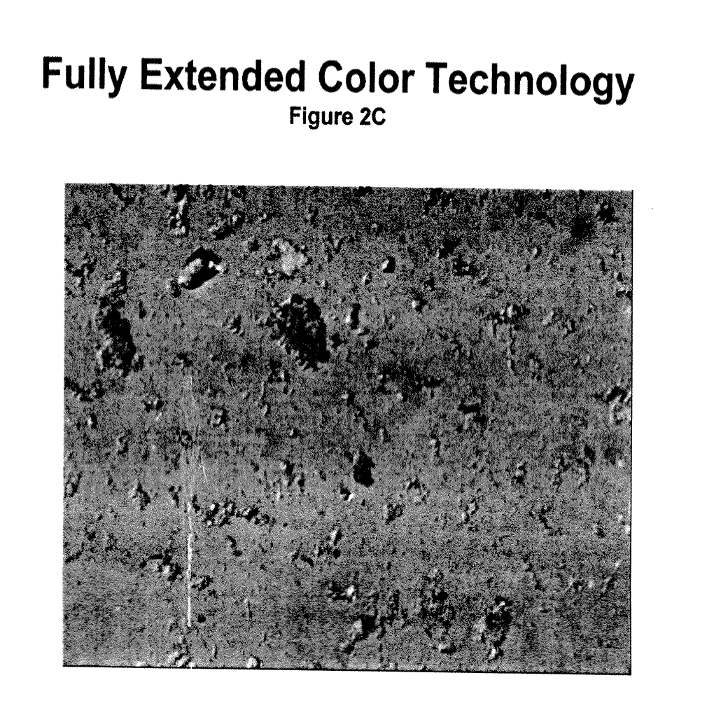

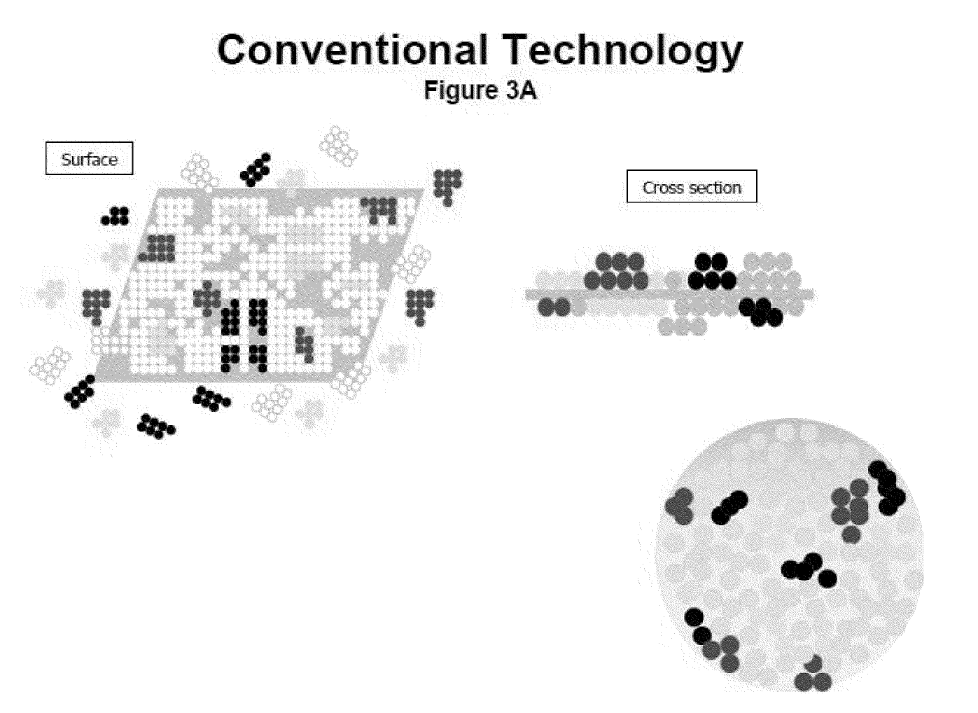

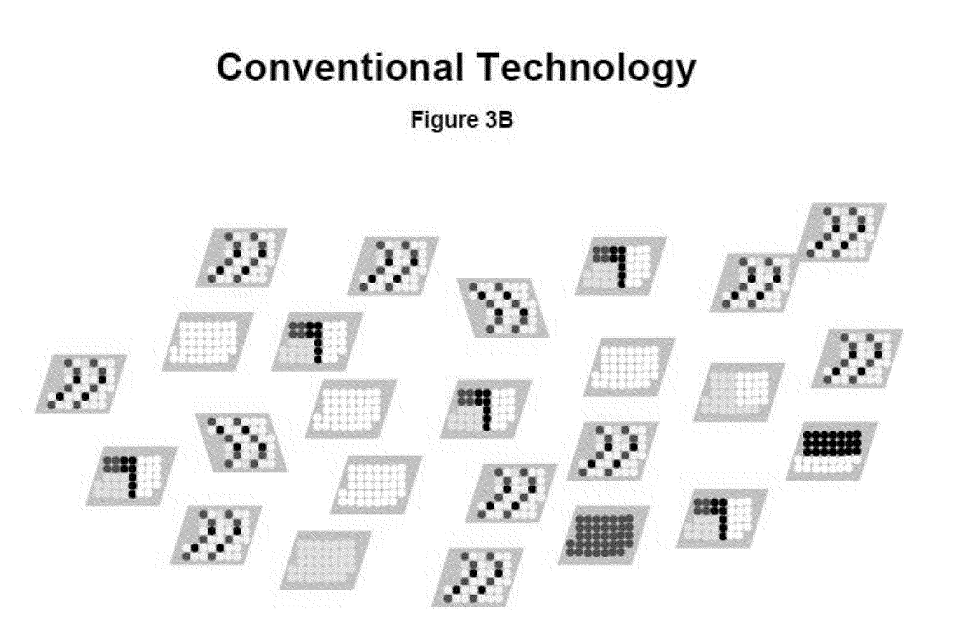

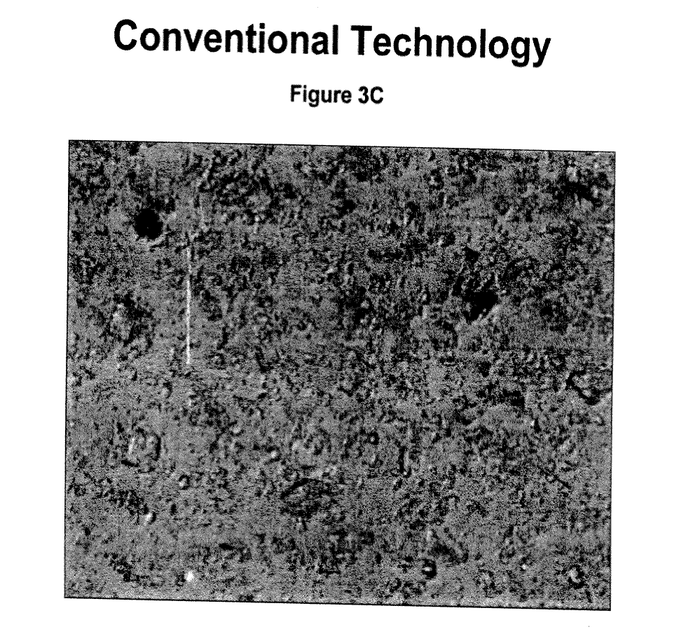

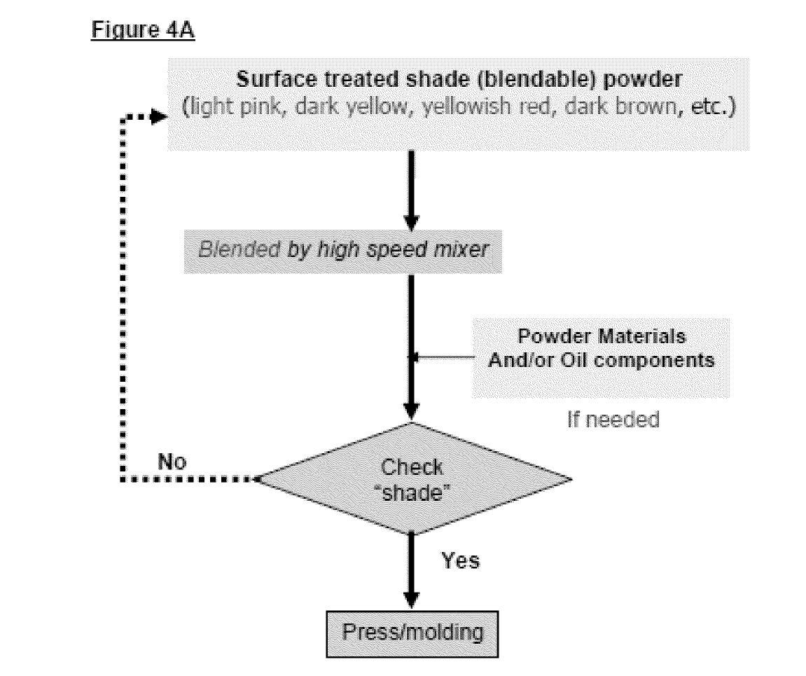

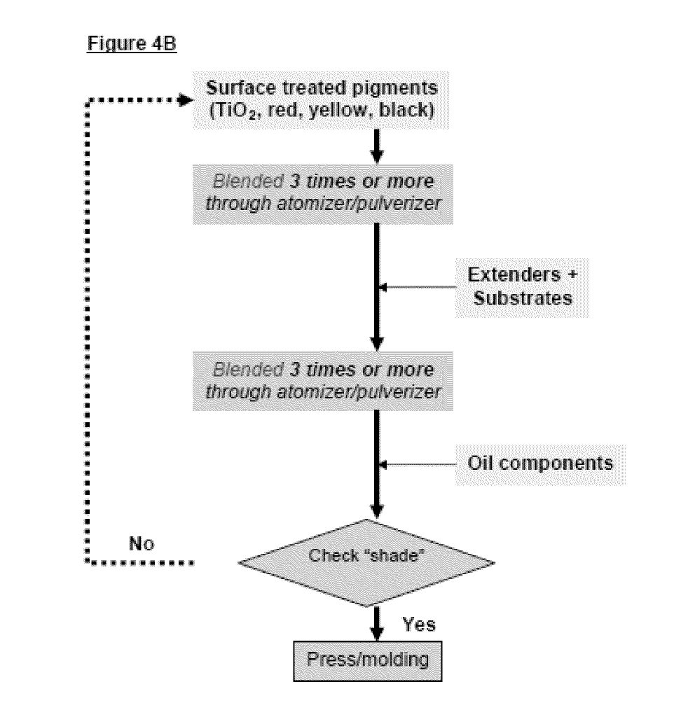

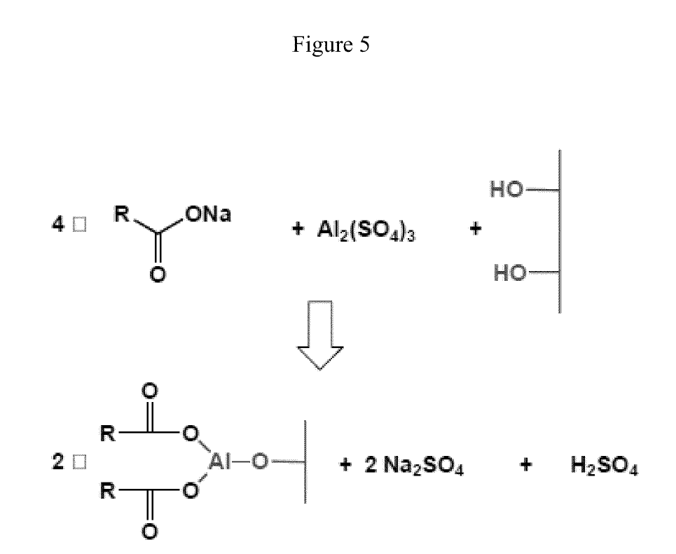

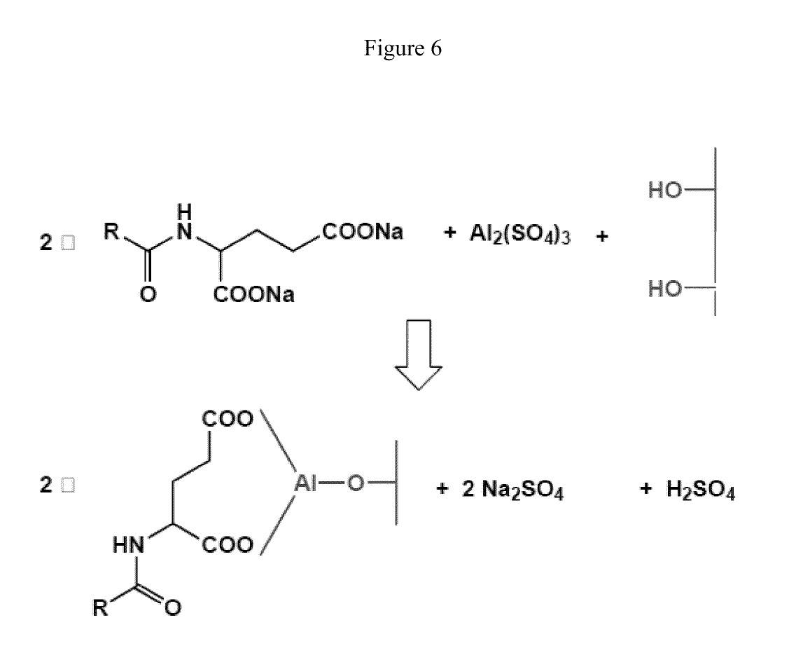

In an exemplary embodiment, a pigment (e.g., deflocculated or deagglomerated pigment) and a substrate are combined to form a mixture. The material is mixed with an aqueous solution (e.g., 50-800% water, based on pigment weight) and dispersed in a liquid slurry. The mixture may include a plurality of different pigments, the pigments in pre-determined amounts or ratios to provide a desired color, shade, hue, chroma (saturation) or lightness (see, for example, Example 2, Tables 1 and 2). A binder, such as an oil (emollient), is optionally added to the slurry (e.g., 0 to 180 parts of oil per 100 part powder). One or more surface treatment agents is then dispersed into the slurry (e.g., about 0.5 to 400 parts surface-treatment agent per 100 parts powder). The surface treatment agent(s) is chemically immobilized by a water soluble compound having a lipophilic or hydrophilic moiety being absorbed onto the surface of the substrate or pigment. As illustrated in FIG. 1, the result is a “composite” substrate/extender having adhered thereto pigment particles. Different colored pigments can be adhered to substrate in an amount that reflects the predetermined amounts or ratios of pigments in the mixture. Pigments can be random and substantially uniformly distributed on the substrate, or be ordered or non-uniformly distributed on the substrate.

Following surface treatment agent immobilization, surface-modified substrate or pigment is optionally dehydrated and rinsed to remove any secondary salts and byproducts, if necessary. A filtered cake is thereby produced which may be further dehydrated to be “powder,” with less than about 10% loss on drying (LOD), for example, 5% LOD, or 3% LOD.

To produce a fully extended color bulk dispersion, the filtered cake or dehydrated powder is re-dispersed into an oil phase. An oil phase may include liquid paraffin, isododecane, squalane, isononyl isononanoate, hexyl laurate, cetyl octanoate, isostearyl neopentanoate, caplylic/capric triglyceride, tocopheryl acetate, retinyl palmitate, ascorbylpalmitate, polydimethylsiloxane, cyclopolydimethylsiloxane, ethylhexyl methoxycinnamate, PEG90 diisostearate, PEG/PPG-8/3 diisostearate, PEG/PPG-8/3 laurate, poly glyceryl-4 diisostearate, etc.

Fully and partially extended color bulk powder and fully and partially extended color bulk dispersion may be included in a cosmetic or makeup product, such as foundations (liquid foundations, loose powders, hot-pour cream foundations), lip sticks, eye shadows, eyeliners, mascaras, lotions, creams, balms, concealers, blushes, rouges, eyebrow liners, lip liners, nail polishes, and sunscreens. They may also be used in personal care (toiletry) products, such as shampoos, conditioners, lotions, deodorants, antiperspirants, moisturizers, balms, soaps and gels; or pharmaceuticals, such as ointments, salves, gels and creams. When fully and partially extended color bulk powder and fully and partially extended color bulk dispersion are in a cosmetic or makeup product or a toiletry product, other typical components used in cosmetic or toiletry products can be added, if desired. For example, cosmetic and makeup products such as lip stick and mascara will often contain various oils, waxes (e.g., Bees wax, carnauba, candellila, ozokerite, etc.) and paraffins.

The present inventors have surprisingly discovered a synergistic UV-activity where a micron-sized (e.g., ˜1-30 μm), coated particle is further coated with an organic UV-active material. This synergistic UV-activity is manifested by particles comprised of non UV-active materials such as clay (kaolin), silica, and nylon. This synergy allows the manufacture of cosmetic formulations having high SPF values, but relatively low concentrations of organic UV-active ingredients.

According to some embodiments, the concentrations of organic UV-active ingredients may be 10% or less (e.g., 9% or less, 8% or less, 7% or less, 6% or less, 5% or less, 4% or less, 3% or less, 2% or less) of the total formulation (wt %). This includes, but is not limited to, concentrations of organic UV-active ingredients between about from 1% to 10%, from 1% to 9.5%, from 1% to 9%, from 1% to 8.5%, from 1% to 8%, from 1% to 7.5%, from 1% to 7%, from 1% to 10%, from 1% to 6%, from 1% to 5.5%, from 1% to 5%, from 1% to 4.5%, from 1% to 4%, from 1% to 3.5%, from 1% to 3%, from 1% to 2.5%, from 1% to 2%, from 1% to 1.5%, from 2% to 10%, from 2% to 9.5%, from 2% to 9%, from 2% to 8.5%, from 2% to 8%, from 2% to 7.5%, from 2% to 7%, from 2% to 10%, from 2% to 6%, from 2% to 5.5%, from 2% to 5%, from 2% to 4.5%, from 2% to 4%, from 2% to 3.5%, from 2% to 3%, from 2% to 2.5%, from 3% to 10%, from 3% to 9.5%, from 3% to 9%, from 3% to 8.5%, from 3% to 8%, from 3% to 7.5%, from 3% to 7%, from 3% to 10%, from 3% to 6%, from 3% to 5.5%, from 3% to 5%, from 3% to 4.5%, from 3% to 4%, from 3% to 3.5%, from 4% to 10%, from 4% to 9.5%, from 4% to 9%, from 4% to 8.5%, from 4% to 8%, from 4% to 7.5%, from 4% to 7%, from 4% to 10%, from 4% to 6%, from 4% to 5.5%, from 4% to 5%, from 4% to 4.5%, from 5% to 10%, from 5% to 9.5%, from 5% to 9%, from 5% to 8.5%, from 5% to 8%, from 5% to 7.5%, from 5% to 7%, from 5% to 10%, from 5% to 6%, and from 5% to 5.5%.

Commercial sunscreens are typically formulated to yield about 1 to 2 SPF units per weight percent (wt %) UV-active ingredient. For example, a typical marketed SPF 50 sunscreen formulations contain a total of approximately 27% UV-active materials such as Octinoxate 7.5%, Oxybenzone 6.0%, Ococrylene 8.0% and Octisalate 5.0%. The present disclosure relates to the term “SPF Index.” SPF Index is herein defined as the numerical ratio of SPF to the concentration of organic UV-actives in weight percent (wt %).

According to some embodiments, the in vitro SPF Index of a formulation may be between 3 and 40, which includes, but is not limited to, between 3 and 40, between 3 and 35, between 3 and 30, between 3 and 25, between 3 and 20, between 3 and 15, between 3 and 10, between 3 and 9, between 3 and 8.5, between 3 and 8, between 3 and 7.5, between 3 and 7, between 3 and 6.5, between 3 and 6, between 3 and 5.5, between 3 and 5, between 3 and 4.5, between 3 and 4, between 3 and 3.5, between 4 and 40, between 4 and 35, between 4 and 30, between 4 and 25, between 4 and 20, between 4 and 15, between 4 and 10, between 4 and 9, between 4 and 8.5, between 4 and 8, between 4 and 7.5, between 4 and 7, between 4 and 6.5, between 4 and 6, between 4 and 5.5, between 4 and 5, between 4 and 4.5, between 5 and 40, between 5 and 35, between 5 and 30, between 5 and 25, between 5 and 20, between 5 and 15, between 5 and 10, between 5 and 9, between 5 and 8.5, between 5 and 8, between 5 and 7.5, between 5 and 7, between 5 and 6.5, between 5 and 6, between 5 and 5.5, between 6 and 40, between 6 and 35, between 6 and 30, between 6 and 25, between 6 and 20, between 6 and 15, between 6 and 10, between 6 and 9, between 6 and 8.5, between 6 and 8, between 6 and 7.5, between 6 and 7, between 6 and 6.5, between 8 and 40, between 8 and 35, between 8 and 30, between 8 and 25, between 8 and 20, between 8 and 15, between 8 and 10, between 8 and 9, between 8 and 8.5, between 10 and 40, between 10 and 35, between 10 and 30, between 10 and 25, between 10 and 20, between 10 and 15, between 10 and 12, between 6 and 16, between 4 and 16, between 6 and 12, and between 4 and 12.

According to some embodiments, the in vitro SPF value of a formulation may be between 10 and 70, which includes, but is not limited to, between 10 and 70, between 10 and 60, between 10 and 50, between 20 and 60; between 25 and 60, between 25 and 50, between 25 and 40, between 30 and 60, and between 30 and 50.

Persons of skill in the art are familiar with several methods for determining SPF values. In-vivo SPF values may be determined in accordance with the procedure set forth in “Proposed Monograph for OTC Drug Products” issued by the Food and Drug Administration, Aug. 25, 1978, Federal Register Volume 43, Number 166, 38206-38269. In vitro SPF values may be determined in accordance with the method of Diffy, B. L. and Robson, J. (1989) (“A new substrate to measure sunscreen protection factor throughout the ultraviolet spectrum,” 40 J. Soc. Cosmet. Chem. 127-133, 1989).

SPF values were determined as proportional to the inverse transmittance at a given wavelength. A layer of Transpore™ (3M, Minneapolis, USA) tape is placed in a single layer on clean approximately 2 mm thick quarts slides. Preferably, an area of at least 40 cm² is applied to enable measurement nine, non-overlapping spots. A minimum of three test samples and at least one control sample was prepared for each sunscreen to be tested. Sample plates were exposed to 280-400 nm UV irradiation in an SPF-290™ Ultraviolet Transmittance Analyzer (Optometrics LLC, Ayer, Mass., USA). The Transpore™ layers were evenly coated with approximately 2 mg/cm² of the appropriate sample or vehicle control was applied to the plates using an FDA-approved finger stall. The plates were weighed on an analytical balance and allowed to incubate for 10 minutes. The sample plates were exposed to UV irradiation as before. Irradiation took place at 9 randomly selected points. SPF values were calculated according to Equation I using software supplied by the manufacturer.

$$SPF = \frac{\int A ( λ ) E ( λ ) d λ}{\int A ( λ ) E ( λ ) / MPF ( λ ) d λ} ,$$

For Equation I, E(λ) is the solar irradiance spectrum at wavelength λ, A(λ) is the erythemal action spectrum at wavelength λ, and MPF(λ) is the monochromatic protection factor at wavelength λ. MPF is roughly the inverse of the transmittance at a given wavelength.

Organic sunscreens, or UV absorbers, for use in the present embodiments include any organic sunscreen which absorbs, blocks or otherwise mitigates ultraviolet radiation. Such sunscreen compositions include, but are not limited to, p-aminobenzoic acid, 2-ethoxyethyl-p-methoxy cinnamate, diethanolamine-p-methoxy cinnamate, digalloyl trioleate, 2,2-dihyroxy-4-methoxybenzophenone, ethyl-4-bis-(hydroxypropyl) aminobenzoate, 2-ethylhexyl-2-cyano-3,3-diphenyl acrylate, ethylhexyl-p-methoxy cinnamate, 2-ethylhexyl salicylate, glyceryl aminobenzoate, 3 3,5-trimethylcyclohexyl salicylate, lawsone with dihydroxyacetone, methyl anthranilate, 2-hydroxy-4-methoxy benzophenone, amyl-p-dimethylamino benzoate, 2-ethylhexyl-p-dimethylamino benzoate, 2-phenylbenzimidazole-5-sulphonic acid, red petroleum, 2-hydroxy-4-methoxybenzophenone-5-sulphonic acid, triethanolamine salicylate, Amiloxate, Ethylhexyl dimethoxybenzylidene dioxoimidazolidine propionate, Ethylhexyl methoxycrylene, and the like, and mixtures thereof.

In addition to the sunscreens recited above, suitable sunscreens or UV absorber for use in the inventive sunscreen compositions are set forth in Sunscreens Monogram, Federal Register, Vol. 58, No. 90, Proposed Rules, p. 28295 (May 12, 1993) Part B. Sunscreens approved by the regulatory authorities of the U.S., EU, Australia, and Japan, and suitable for purposes disclosed herein may include: PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octocrylene, Octinoxate, Octisalate, Sulisobenzone, Trolamine salicylate, Avobenzone, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Sodium Dihydroxy Dimethoxy Disulfobenzophenone (Benzophenone-9), Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate and mixtures thereof.

Non-limiting preferred sunscreens or UV absorbers include: Octinoxate (ethylhexyl methoxycinnamate, Oxybenzone (benzophenone-3), mentyl anthranilate, octocrylene, homosalate, octisalate, avobenzone, and mixtures thereof.

The present disclosure provides general procedures for the surface treatment of the cosmetic powder. In an embodiment, a cosmetic powder is combined in a vessel with an amount of water or a mixture of water and organic solvent like ethanol, isopropanol, etc. to form a slurry. The cosmetic powder may be a single material or may be a mixture of the various substrate materials disclosed herein. Surface-treatment agents, such as disclosed in paragraphs 0046-0059 are added to the slurry and mixed. At least one polyvalent metal salt such as, but not limited to, aluminum sulfate, aluminum chloride, magnesium sulphate, magnesium sulfate, calcium chloride, calcium sulfate, is added to immobilize the treating agents to the powder surface.

A liquid organic UV-active material is added to the slurry above. The UV-active may be a single substance or may be a combination of substances. Where the UV-active is a room-temperature solid, such as oxybenzone or avobenzone or a similar material, the solid should be dissolved in a liquid organic solvent. Preferably, the organic solvent should, itself, be a UV-active material such as, but not limited to, octocrylene or octinoxate.

In an embodiment, the UV-active material may be added to the cosmetic powder prior to the addition of the polyvalent metal salt. In an embodiment, the UV-active material may be added to the cosmetic powder after the addition of the polyvalent metal salt. In an embodiment, the UV-active material may be added to the cosmetic powder simultaneously with the addition of the polyvalent metal salt.

The covalent surface-treating reaction is allowed to proceed to completion and a “wetcake,” comprising a surface-treated cosmetic powder with bound UV-active, is separated from un-reacted materials. The separation may be effected by means including, but not limited to centrifugation and Buchner filtration.

The wet cake may be dispersed into water by mixing with homogenizer, disperser, propeller mixer, or other mechanical means as is known in the art. Additional cosmetic materials are to be added to stabilize the system and to adjust the texture as a sunscreen formula. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

The “wetcake” residue resulting from Buchner filtration or centrifugation comprises a hydrophobic, surface-modified cosmetic powder dampened with residual water. The hydrophobic coating causes and the UV-active sunscreen oils to adhere to the surface-modified cosmetic powder. The treatment acts as a surfactant layer between water and the oils and cosmetic powder.

According to an embodiment, the wetcake may be at least partially dried. Drying may be at a temperature of from about 105° C. to about 120° C. for from about 1 to about 10 hours. Drying may be continued until a desired degree of dryness is obtained.

Alternatively, the various ingredients may be blended by “kneading.” The knead-blend method allows for the use of substantially less water than is required to form a wetcake. Knead-blending also produces a much drier product (knead-blend) compared to a wetcake.

A knead-blend may be formed by moistening a cosmetic powder with a solvent. The cosmetic powder may be a single substance or may be a mixture of several substances. The solvent may be water or a mixture of water and an organic solvent. The organic solvent is preferably a lower alcohol such as, but not limited to, ethanol or isopropanol. The organic solvent may be a mixture of solvents.

A surface-treatment agent, or a mixture thereof, is added to the moistened cosmetic powder and blended. Suitable surface-treatment agents are disclosed in paragraphs 0046-0059.

At least one polyvalent metal salt such as, but not limited to, aluminum sulfate, aluminum chloride, magnesium sulphate, magnesium sulfate, calcium chloride, calcium sulfate, is added to immobilize the treating agents to the powder surface.

A liquid organic UV-active material is added to the slurry above. The UV-active may be a single substance or may be a combination of substances. Where the UV-active is a room-temperature solid, such as oxybenzone or avobenzone or a similar material, the solid should be dissolved in a liquid organic solvent. Preferably, the organic solvent should, itself, be a UV-active material such as, but not limited to, octocrylene or octinoxate.

The knead-blend, comprised of surface-treated cosmetic powder with bound UV-active, is retrieved as a moist composition.

In an embodiment, the UV-active material may be added to the cosmetic powder prior to the addition of the polyvalent metal salt. In an embodiment, the UV-active material may be added to the cosmetic powder after the addition of the polyvalent metal salt. In an embodiment, the UV-active material may be added to the cosmetic powder simultaneously with the addition of the polyvalent metal salt.

In an embodiment, the knead-blend may be at least partially dried. Drying may be at a temperature of from about 105° C. to about 120° C. for from about 1 to about 10 hours. Drying my be continued until a desired degree of dryness is obtained.

In an embodiment the dried surface-treated material with bound UV-actives may be dispersed into cosmetic formulations such as anhydrous systems or water-in-oil emulsions. In an embodiment the dried surface-treated material with bound UV-actives may be formulated as a powder system such as, but not limited to a pressed foundation powder or a loose powder. Other cosmetic ingredients may be assed as appropriate. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

The wetcake or the knead-blend produced by any of the above methods may be optionally oven dried. Drying typically is performed overnight at 105° C. This method removes most or all of the retained water. The “dried” product consists of treated cosmetic powder containing the sunscreen oils. The lipophilic nature of the surface coating and the sunscreen actives allow for a stable system.

In an embodiment, the optionally-dried wetcake and/or knead-blend may be emulsified to form a stable oil-in-water (OW) system. An amount of water is added to the optionally-dried wetcake and/or knead-blend and mixed with a homogenizer, dispersion blade, or prop. The choice of blending instrument may depend on the amount of energy required as is apparent to a person of skill in the art. A rheological modifier (thickening agent) is added with continued mixing. The rheological modifier may be Simulgel™ NS or Dow RM2051™. Addition of the rheological modifier produces a final product having a creamy lotion texture.

Without being bound by theory, the inventors believe that it is the quality of the dispersion, specifically the treated cosmetic powder combined with the UV active oils that produce a “blanket” of coverage upon application, which provides the basis for the enhanced SPF.

In an embodiment, the optionally-dried wetcake and/or knead-blend may be emulsified to form a stable water-in-oil (WO) system. The optionally-dried wetcake and/or knead-blend may be homogenized with typical oils, such as, but not limited to isononyl isononanoate (“ININ”), cyclomethicone (cyclopentadimethylsiloxane, “D5”), isododecane, and emulsifiers, such as, but not limited to ABIL WE09 (a blend of polyglyceryl-4 isostearate (and) cetyl PEG/PPG-10/1 dimethicone (and) hexyl laurate).

The wetcake or knead-blend may be formulated with other cosmetic ingredients. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

Fully and partially extended color bulk powders and fully and partially extended color bulk dispersions and intermediate products thereof can be included in containers and kits, optionally including instructions for changing a shade of a fully or partially extended color bulk powder or fully or partially extended color bulk dispersion, or formulating a fully extended color bulk powder or fully extended color bulk dispersion in a cosmetic or makeup, personal care or pharmaceutical product. Specific non-limiting examples of containers and kits include a pan or bottle which contains a fully extended color bulk powder or fully extended color bulk dispersion. with at least one well therein.

A container or kit optionally includes “packaging material,” which refers to a physical structure housing a container or kit, or a component(s) of the container or kit. The packaging material can be made of material commonly used for such purposes. A container or kit can include a label or packaging insert with appropriate instructions, for example. Instructions may be on “printed matter,” e.g., on paper or cardboard within the container or kit, or on a label affixed to the container or kit. Instructions may be provide on audio or video medium, such as an a computer readable medium, for example, floppy diskette or hard disk, optical CD such as CD- or DVD-ROM/RAM, magnetic tape, electrical storage media such as RAM and ROM and hybrids of these such as magnetic/optical storage media.

Specific non-limiting examples of containers and kits suitable for a bulk color powder and dispersion include pans, bottles, jars, vials, and tubes. Materials suitable for pans, bottles, jars, vials, and tubes include metal, glass or a polyolefin. Exemplary metals include iron (steel) and aluminum. Exemplary polyolefins include polystyrene, polypropylene, polyethylene, and polybutylene. Additional specific non-limiting examples of containers and kits include pouches, boxes, cartons and drums.

Containers and kits may be sealed. Containers and kits may include multiple (two or more) types of fully or partially extended color bulk powder, fully or partially extended color bulk dispersion or intermediate thereof. For example, a container such as a pan can include two or more wells, each of which contain a different color, shade, hue, chroma (saturation) or lightness of fully extended color bulk powder, fully extended color bulk dispersion, or an intermediate thereof. Thus, a container may contain wells, each of which have deposited thereon 1) a light pink shade fully extended color bulk powder; 2) a dark yellow shade fully extended color bulk powder; 3) a yellowish-red shade fully extended color bulk powder; and 4) a dark brown shade fully extended color bulk powder, so that when two or more of the shades are combined a different color, shade, hue, chroma (saturation) or lightness is produced. In another particular non-limiting example, a container or kit can include multiple packages each of which contains 1) a fair shade fully extended color bulk powder; 2) a golden shade fully extended color bulk powder; 3) a rose shade fully extended color bulk powder; and 4) a bark shade fully extended color bulk powder, in individual packages.

Unless otherwise defined, all technical and scientific terms used herein have the same meaning as commonly understood by one of ordinary skill in the art to which this application relates. Although methods and materials similar or equivalent to those described herein can be used in the practice or testing of the disclosed embodiments, suitable methods and materials are described herein.

All publications, patents, and other references cited herein are incorporated by reference in their entirety. In case of conflict, the present specification, including definitions, will control.

As used herein, singular forms “a,” “and,” and “the” include plural referents unless the context clearly indicates otherwise. Thus, for example, reference to “a pigment” includes a plurality of pigments, reference to “a substrate” can include a plurality of substrates and reference to “an extender, emulsifier, surfactant, oil, etc.” can include a plurality of extenders, emulsifiers, surfactants, oils, and so forth.

As used herein, all numerical values or numerical ranges include whole integers and fractions thereof within or encompassing such ranges unless the context clearly indicates otherwise. Thus, for example, reference to values such as 0 to 25% includes 0% to 5% (i.e., 1, 2, 3, 4, 5%, or 1.1, 1.2, 1.3, 1.4, 1.5%, etc.), 10% to 20% (10, 11, 12, 13, 14%, etc., or 10.1, 10.2, 10.3, 10.4, 10.5, etc.), and so forth.

Reference to specific amount of a given ingredient or component in a color bulk powder, color bulk dispersion or intermediate thereof (e.g., a pigment, substrate, extender, oil, binder, treatment agent, etc.), include variations within about 1 to 20%, or 1 to 10%, or 5 to 10%, unless indicated otherwise. The term “about” typically refers to a value with about +/−1 to 10%, or 5 to 10% of the reference value.

As used herein, the term “QS,” as is accustomed in the art, means a “sufficient quantity” to obtain the desired functionality. For a fragrance, the functionality is typically obtained using from about 0.05 to 1.0 wt %; for a preservative, the functionality is typically obtained using from about 0.01 to 1.0 wt %.

The present embodiments are generally disclosed using affirmative language to describe the numerous embodiments. Some embodiments are described in which particular subject matter is excluded, in full or in part. For example, one or more powder materials (e.g., substrates, pigments, pigment extender, etc.), surface treatment agents, binders (e.g., oils), emulsifiers, preservatives and fragrances can be specifically excluded in a composition or method of the invention. Thus, even though some embodiments are generally not expressed in terms of what the embodiments do not include, compositions and methods disclosed herein include embodiments in which one or more powder materials (e.g., substrates, pigments, extenders, etc.), surface treatment agents, binders (e.g., oils/emollients), emulsifiers, preservatives, fragrances, etc., are excluded.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of a general procedure for preparing hydrophobically modified powder materials with fully extended color/shade, and bulk dispersion of hydrophobically modified powder materials with fully extended color/shade.

Powder material is mixed with 50 to 800% (based upon weight of the pigment) water and dispersed to make a slurry. An aqueous solution of one or more surface-treatment agents, for example, a water-soluble alkali metal salt of a fatty acid or an acyl amino acid, is added to the slurry and dispersed (0.5 to 400 parts surface-active agent per 100 parts powder). One to two chemical equivalents of a water-soluble salt of a polyvalent metal, such as an alkaline earth metal, calcium, magnesium, aluminum, titanium, zinc, or zirconium sulfate or chloride may be added to a powder material to assist in linking a functional group of the surface-treatment agent to the surface of the particles of the powder material. An oil (0 to 180 parts of oil per 100 part powder) is optionally introduced. The result is an oil coated or non oil coated, surface-modified powder material.

The oil coated or non oil coated surface-modified powder material (cake) is then optionally dehydrated, for example, using a filter press, centrifuge, freeze-drying, etc. Prior to or following dehydration, oil coated or non oil coated surface-modified powder material is optionally rinsed with water (e.g., purified, distilled or de-ionized), a water-organic solvent, and an alcohol mixture, to remove any secondary salts or byproducts, if necessary. If desired, the filtered material (cake) is further dehydrated with heat or drying to a “powder” until the moisture content reaches a desired target value, for example, 10%, or 5%, or less (e.g., 3%), loss on drying (LOD). Typical conditions for drying, which depends in part on batch size, are heating in an oven to about 100° C. for about 2 hours. After cooling, the material can be further processed, for example, deflocculated. For a dispersion, filtered material (cake) or powder can be re-dispersed into an oil phase, water in oil, oil in water, or water in silicone system to produce a suspension/emulsion.

### Example 2

This example describes particular non-limiting examples of fully extended color bulk powders, and fully extended color bulk powder dispersions (water in oil emulsions).

Exemplary extended color bulk powders, and fully extended color bulk powder dispersions, a set forth in Tables 1 and 2, were prepared as follows:

Powder Formulations 1 through 5 were Prepared in Accordance with Following Procedure.
1. Fill a tank with warm water and start high shear mixer at a speed above 6,000 rpm.
2. Add pigments and homogenize for a period of time to mix.
3. Reduce mixing speed and add extenders, substrates and other agents while continuing to mix for a period of time to allow the components to blend.
4. Add cosmetic oil, and mix.
5. Add aluminum sulfate solution to initiate surface treatment of powder materials.
6. Filter and rinse the powder with an amount of water to remove any salt byproduct (check conductivity).
7. Dry the wet product in an oven.
8. Pulverize the dried product with 0.020 screen, and test the powder for color extension.

Formulations 14 and 17 were prepared with Powder Formulations 2 through 5 as described above, by using a planetary mixer for 10 minutes. This powder was tested for color extension.

TABLE 1
Fully extended color bulk powder

| | | Formula 1 | Formula 2 | Formula 3 | Formula 4 | Formula 5 |
|---|---|---|---|---|---|---|
| Surface | Sodium myristoyl | — | — | — | — | 1.50 |
| Treatment Agent | sarcosinate | | | | | |
| | Disodium stearoyl | 1.00 | 1.00 | — | 2.00 | — |
| | glutamate | | | | | |
| | Potassium palmitate | — | — | 1.50 | — | — |
| | Potassium or aluminum | 1.50 | 1.50 | — | — | 3.50 |
| | myristate | | | | | |
| | Triethoxy capryl silane | 2.00 | 3.00 | 1.50 | — | 2.00 |
| | Dimethicone | — | — | 1.50 | 2.50 | — |
| Pigments | Titanium dioxide | 5.00 | 10.00 | 0.50 | 0.50 | 0.50 |
| | Yellow iron oxide | 0.99 | 0.15 | 2.70 | 8.00 | 4.25 |
| | Red iron oxide | 0.33 | 0.10 | 6.00 | 0.80 | 4.90 |
| | Black iron oxide | 0.18 | 0.03 | 0.33 | 0.30 | 9.15 |
| | Pearl pigments | 0.50 | — | — | — | — |
| Substrates | Talc | Balance to | Balance to | Balance to | Balance to | Balance to |
| | | 100.00 | 100.00 | 100.00 | 100.00 | 100.00 |
| | Mica | 20.00 | 20.00 | 15.0 | 15.0 | 20.0 |
| | Sericite | 25.00 | 25.00 | 25.0 | 25.0 | 25.0 |
| | Silica bead | 1.00 | — | — | — | 3.00 |
| | Aluminum calcium | — | 1.00 | — | 2.00 | — |
| | sodium silicate | | | | | |
| | Boron nitride | — | — | — | 3.00 | 5.00 |
| | Nylon bead | — | — | 3.00 | — | — |
| | PMMA | — | — | — | — | — |
| | Ultrafine TiO₂ | — | 5.00 | — | — | — |
| | Ultrafine ZnO | — | 3.00 | — | — | — |
| Cosmetic Oil | Cetyl octanoate | — | 2.50 | — | — | — |
| | Dimethicone | — | — | — | 3.00 | 3.00 |
| | Cetyl dimethicone | — | — | — | 2.50 | — |
| | Caprylic/capric | — | — | — | 1.00 | — |
| | triglyceride | | | | | |
| | Diisostearyl maleate | 3.00 | 3.50 | — | — | 2.00 |
| | Squalane | 2.00 | — | — | — | 2.00 |
| | Tocopheryl acetate | — | 0.20 | 0.50 | 0.20 | — |
| | Octylmethoxycinnamate | — | — | 6.50 | — | — |
| | Preservatives | QS | QS | QS | QS | QS |
| | Fragrance | QS | QS | QS | QS | QS |

TABLE 2
Fully extended color bulk water in oil (W/O) emulsions

| | | Formula 6 | Formula 7 | Formula 8 | Formula 9 | Formula 10 |
|---|---|---|---|---|---|---|
| | | O/W | O/W | O/W | W/O | W/O |
| Surface Treatment | Sodium myristoyl | 5.00 | — | — | 2.50 | 5.00 |
| Agents | sarcosinate | | | | | |
| | Disodium stearoyl | — | 2.50 | 2.50 | — | — |
| | glutamate | | | | | |
| | Potassium palmitate | — | 2.50 | 2.50 | — | — |
| | Potassium myristate | — | — | — | 2.50 | — |
| | Zinc gluconate | 5.00 | 5.00 | 5.00 | 7.00 | 5.00 |
| Pigments | Titanium dioxide | 7.00 | 15.00 | 15.00 | 8.00 | 15.00 |
| | Yellow iron oxide | 0.88 | 3.24 | 3.24 | 2.95 | 3.24 |
| | Red iron oxide | 0.29 | 0.65 | 0.65 | 0.55 | 0.65 |
| | Black iron oxide | 0.13 | 0.36 | 0.36 | 0.25 | 0.36 |
| | Pearl pigments | — | 0.50 | — | — | — |
| Substrates | Talc | — | — | — | — | — |
| | Kaolin | 5.00 | — | — | — | — |
| | Mica | 1.00 | 2.00 | 2.00 | 5.00 | 5.00 |
| | Sericite | — | — | — | — | — |
| | Silica bead | — | 0.50 | — | — | — |
| | Aluminum calcium | — | — | — | 0.50 | 0.50 |
| | sodium silicate | | | | | |
| | Nylon bead | 2.00 | 2.00 | 2.00 | — | — |
| | PMMA | 2.00 | — | — | — | — |
| | Ultrafine TiO₂ | — | 6.00 | 10.00 | 7.00 | 10.00 |
| | Ultrafine ZnO | — | — | — | 3.00 | — |
| Cosmetic Oil | Isododecane | 10.55 | 7.00 | — | — | — |
| | Isononyl | 4.00 | 4.00 | 4.00 | — | — |
| | isononanoate | | | | | |
| | Cholesteryl/Behenyl/ | 1.00 | — | — | — | — |
| | Octyldodecyl lauroyl | | | | | |
| | Glutamate | | | | | |
| | Caprylic/capric | — | 1.00 | — | — | — |
| | triglyceride | | | | | |
| | Diisostearyl maleate | — | 3.00 | 3.00 | — | — |
| | Behenyl alcohol | 0.50 | 0.50 | 0.75 | — | — |
| | Tocopheryl acetate | 0.10 | 0.10 | 0.10 | 0.10 | 0.10 |
| | Diphenyldimethicone | — | — | 1.00 | 1.00 | 1.00 |
| | Dimethicone | — | — | 2.00 | — | — |
| | Cyclomethicone | — | — | 10.00 | 15.00 | 15.00 |
| | Ethylhexyl | 5.00 | — | — | — | — |
| | methoxycinnamate | | | | | |
| Emulsifiers | Cetyl dimethicone | — | — | — | 0.50 | — |
| | copolyol | | | | | |
| | Polygyceryl-4 | — | — | — | — | — |
| | isosteatrate | | | | | |
| | Glyceryl stearate | 1.25 | 1.50 | 1.50 | — | — |
| | (and) PEG-100 | | | | | |
| | stearate | | | | | |
| | Cetyl alcohol (and) | 0.75 | 1.00 | 1.00 | — | — |
| | Dicetl phosphate | | | | | |
| | (and) Ceteth-10 | | | | | |
| | Phosphate | | | | | |
| | Isostearic acid | 1.20 | — | — | — | — |
| | Preservatives | QS | QS | QS | QS | QS |
| | Fragrance | QS | QS | QS | QS | QS |
| Water Phase | Gum | 0.20 | 0.20 | 0.20 | 0.10 | 0.10 |
| | Glycerin | 7.00 | 7.00 | 7.00 | 5.00 | 9.00 |
| | Butylene glycol | 4.00 | 4.00 | 4.00 | 6.00 | 2.00 |
| | Water | Balance to | Balance to | Balance to | Balance to | Balance to |
| | | 100.00 | 100.00 | 100.00 | 100.00 | 100.00 |

| | | Formula 11 | Formula 12 | Formula 13 |
|---|---|---|---|---|
| | | W/O | W/O | W/O |
| Surface Treatment | Sodium myristoyl | 5.00 | 3.00 | — |
| Agents | sarcosinate | | | |
| | Disodium stearoyl | — | — | 5.00 |
| | glutamate | | | |
| | Potassium myristate | — | 2.00 | — |
| | Zinc gluconate | — | — | — |
| | Dimethicone | 3.00 | 3.00 | 3.00 |
| Pigments | Titanium dioxide | 8.00 | 8.00 | — |
| | Yellow iron oxide | 1.10 | 1.10 | — |
| | Red iron oxide | 0.35 | 0.35 | — |
| | Black iron oxide | 0.20 | 0.20 | — |
| | Brown iron oxide | — | — | 5.00 |
| | Pearl pigments | — | — | — |
| Substrates | Talc | — | — | 10.00 |
| | Kaolin | — | — | — |
| | Mica | — | — | 1.50 |
| | Sericite | — | — | 2.00 |
| | Silica bead | — | — | 1.00 |
| | Aluminum calcium | — | — | — |
| | sodium silicate | | | |
| | Nylon bead | — | — | — |
| | Boron nitride | 8.00 | 8.00 | 0.50 |
| | Ultrafine TiO₂ | — | 5.00 | — |
| | Ultrafine ZnO | — | — | — |
| Cosmetic Oil | Isododecane | 5.00 | 15.00 | — |
| | Isononyl isononanoate | 20.00 | 15.00 | 5.00 |
| | Dimethicone | — | — | 10.00 |
| | Cyclomethicone | — | — | 10.00 |
| | Ethylhexyl | 7.50 | — | — |
| | methoxycinnamate | | | |
| Emulsifiers | Cetyl dimethicone | — | 0.50 | 0.50 |
| | copolyol | | | |
| | Polygyceryl-4 isosteatrate | 3.00 | 3.00 | 2.50 |
| | Quaternium-18 hectorite | — | — | 0.50 |
| | or Quaternium-18 | | | |
| | bentonite | | | |
| | Preservatives | QS | QS | QS |
| | Fragrance | QS | QS | QS |
| Water | Gum | 0.20 | 0.20 | 0.10 |
| Phase | Glycerin | — | — | — |
| | Butylene glycol | 4.00 | 4.00 | 4.00 |
| | Water | Balance to | Balance to | Balance to |
| | | 100.00 | 100.00 | 100.00 |

TABLE 3
Formulas for 4 different shades of fully extended color bulk powder, which
can be blended by conventional high speed mixer to create changes in shade

| | | Light | Dark | | Dark |
|---|---|---|---|---|---|
| | | pink shade | yellow shade | Yellowish | brown shade |
| | | Light | yellow | red shade | dark |
| | | skin tone color | brown color | Red color | brown color |
| color pigments | TiO2 | Rich 80 | Typical 28 | Typical 12 | Typical 1 |
| 5 to 20% wt | yellow iron oxide | Typical 16 | Rich 60 | Typical 24 | Typical 15 |
| | red iron oxide | Typical 3 | Typical 8 | Rich 60 | Typical 24 |
| | black iron oxide | Typical 1 | Typical 4 | Typical 4 | Rich 60 |
| | ultramarine, etc. | Typical QS | Typical QS | Typical QS | Typical QS |
| Substrates | talk, mica, sericite, | typical | typical | typical | typical |
| 0~95% | kaolin, silica bead, | | | | |
| | nylon powder, | | | | |
| | functional powders | | | | |
| surface | fatty acids, acyl | typical | typical | typical | typical |
| treatment | amino acids, | | | | |
| 0.5~10% wt | silanes, . . . etc. | | | | |
| incorporated | dimithicone, esters, | typical | typical | typical | typical |
| emollient | ethers, glycerides, | | | | |
| 0~25% | hydrocarbons, etc. | | | | |
| additional oily | dimithicone, esters, | typical | typical | typical | typical |
| components | ethers, glycerides, | | | | |
| 0~25% | hydrocarbons, etc. | | | | |

Fully extended color bulk powders, as exemplified by four shades in Table 3, can be mixed in various amounts and proportions to produce a desired color. As an example, four shades of pigment are mixed at the ratios indicated in Table 4 below by “high speed blade mixer” or “atomizer” for certain period, which depends upon batch size. For “light skin tone,” a typical mixing ratio of TiO2, yellow, red and black is about 80%, 16%, 3% and 1% (total is 100). Occasionally, a color like ultramarine is used in relatively small amounts. For “yellow brown color,” yellow is used at 60% and other pigments are used at 28% for TiO2, 8% for red and 4% for black (total is 100).

After mixing, the blended fully extended color bulk powders can be pressed into pans. The color of the cake is already fully extended. Rubbing the cake surface with a sponge or finger does not substantially change the color, and the same color is applied onto the skin. Rubbing the cake surface with a moist sponge also does not substantially change the color.

TABLE 4
Sample mixing ratios for fully extended color bulk powders

| | Formu- | Formu- | Formu- | Formu- |
|---|---|---|---|---|
| | la 14 | la 15 | la 16 | la 17 |
| Light pink | 65.00 | 55.00 | 50.00 | 5.00 |
| shade Formula 2 | | | | |
| Dark yellow | 20.00 | 20.00 | 25.00 | 8.00 |
| shade Formula 4 | | | | |
| Yellowish red | 10.00 | 15.00 | 18.00 | 10.00 |
| shade Formula 3 | | | | |
| Dark brown | 5.00 | 10.00 | 7.00 | 77.00 |
| shade Formula 5 | | | | |

TABLE 5
Formulas for 4 different shades of fully extended color bulk dispersion,
which can be blended by regular homogenizer creating the shades

| | | Light | Dark | | Dark |
|---|---|---|---|---|---|
| | | pink shade | yellow shade | Yellowish | brown shade |
| | | Light skin | yellow | red shade | dark |
| | | tone color | brown color | Red color | brown color |
| color pigments | TiO2 | Rich 80 | Typical 28 | Typical 12 | Typical 1 |
| 5~20% wt | yellow iron oxide | Typical 16 | Rich 60 | Typical 24 | Typical 15 |
| | red iron oxide | Typical 3 | Typical 8 | Rich 60 | Typical 24 |
| | black iron oxide | Typical 1 | Typical 4 | Typical 4 | Rich 60 |
| | ultramarine, etc. | Typical QS | Typical QS | Typical QS | Typical QS |
| substrates | talk, mica, sericite, | typical | typical | typical | typical |
| 1~95% | kaolin, silica bead, | | | | |
| | nilon powder, | | | | |
| | functional powders | | | | |
| surface | fatty acids, acyl | typical | typical | typical | typical |
| treatment | amino acids, | | | | |
| 0.5~20% wt | silanes, . . . etc. | | | | |
| incorporated | dimithicone, esters, | typical | typical | typical | typical |
| emollient | ethers, glycerides, | | | | |
| 0~25% | hydrocarbons, etc. | | | | |
| additional oily | dimithicone, esters, | typical | typical | typical | typical |
| components | ethers, glycerides, | | | | |
| 0~25% | hydrocarbons, etc. | | | | |
| Emulsifiers/ | surfactants, | typical | typical | typical | typical |
| suspending | thickeners, etc. | | | | |
| agents 0~8% | | | | | |

| | | | | |
|---|---|---|---|---|
| Water | typical | typical | typical | typical |

[Note]
If “water” = “0”, it becomes “oil dispersion”
If “oily components” = “0”, it becomes “water dispersion”
If both “water” and “oily components” ≢ “0”, it becomes a emulsion, which can be categorized as either O/W or W/O.

To obtain a desired skin shade, simply mix the 4 shade dispersions in the desired proportions with an homogenizer. The color strength (saturation) can be adjusted by dilution, for example, with “oil phase” and “water phase” or addition of other cosmetic ingredients, if needed.

Fully extended color powders were compared to conventional powders, as described in U.S. Pat. No. 5,968,531. Comparative Formulas 1 and 2 set forth in Tables 6 and 7 were prepared as described in U.S. Pat. No. 5,968,531, incorporated herein by reference in its entirety. Example 1 in U.S. Pat. No. 5,968,531 provides as follows:

“Mixture of 200 g of talc with mean particle size on volume basis by laser diffraction of 8.2 micron (Soft talc from Miki America, Inc.) and 67 g of micronized titanium dioxide with mean particle size on volume basis by laser diffraction of 0.042 micron (UFTR from U.S. Cosmetics Co.) was mixed with 5.3 g of USP grade zinc stearate with meting point of between 115° C. and 125° C. (Witco Co.) in Mizuho MP-5 mixer for 30 minutes. The tip speed of the blades was set at 40 m/sec and the temperature of the powders inside of the mixer at the end of the process was 120° C., The sample had low oil absorption, good sheerness and slip, and uniform attachment of the micronized titanium dioxide on the surface of the talc was confirmed by SEM observation . . . . To test the stability of the composite, the composite sample was stirred in the water in the beaker to see if micronized titanium dioxide can be detached. However, water was clear after 50 stirs by spoon, and the composite sample showed no disintegration in water. Additional test for the stability of the sample was carried out. The mixture of 0.5 grams of the sample in 20 ml of ethyl alcohol in a test tube was put in the ultrasonic cleaner (Branson 1200) for five minutes. After four hour the clarity of the ethyl alcohol was observed for the detachment due to the ultrasonic energy and subsequent suspension of the fine particles of metal oxide in ethyl alcohol. The solution was clear after four hours and therefore the result confirmed the attachment of the fine particles of titanium dioxide over the talc. The sample was further tested for its UV transmittance by UV/visible range spectrometer (Shimadzu UV 160) for its UV screening effect.”

TABLE 6
Comparative Formula 1, which corresponds
to Formula 3 in U.S. Pat. No. 5,968,531

| Ingredients | % wt |
|---|---|
| Example 1 in the U.S. Pat. No._5,968,531 | 35.00 |
| SAT-TRI-77891 (Titanium Dioxides (and) | 10.0 |
| Dimethicone) | |
| SAT-Y-77492 (Iron Oxides (and) Dimethicone) | 2.50 |
| SAT-R-77491 (Iron Oxides (and) Dimethicone) | 0.80 |
| SAT-B-77499 (Iron Oxides (and) Dimethicone) | 0.30 |
| SAT-Mica (Mica (and) Dimethicone) | 7.00 |
| SAT-Sericite (Seericite (and) Dimethicone) | 20.00 |
| SAT-Talc (Talc (and) Dimethicone) | Balance to 100.00 |
| PMMA | 3.00 |
| Silica | 2.00 |
| Caprylic/capric triglyceride | 4.00 |
| Dimethicone 20 cst. | 8.20 |
| Preservative | QS |
| Fragrance | QS |

TABLE 7
Comparative Formula 2 consists of monochromatic
color pigments of which talc is an extender

| Ingredients | % wt |
|---|---|
| FN-TRI-77891 (Talc (and) Titanium Dioxide | 7.15 |
| (and) C8-18 Fluoroalcohol Phosphate | |
| (and) Aluminum Hydroxide) | |
| FN-Y-77492 (Talc (and) Iron Oxides (and) C8-18 | 1.43 |
| Fluoroalcohol Phosphate (and) Aluminum Hydroxide) | |
| FN-R-77491 (Talc (and) Iron Oxides (and) C8-18 | 0.50 |
| Fluoroalcohol Phosphate (and) Aluminum Hydroxide) | |
| FN-B-77499 (Talc (and) Iron Oxides (and) C8-18 | 0.29 |
| Fluoroalcohol Phosphate (and) Aluminum Hydroxide) | |
| SAT-Mica (Mica (and) Dimethicone) | 15.00 |
| SAT-Sericite (Seericite (and) Dimethicone) | 25.00 |
| SAT-Talc (Talc (and) Dimethicone) | Balance to 100.00 |
| SAT-Silica (Silica (and) Dimethicone) | 3.00 |
| Boron Nitride | 2.50 |
| Caprylic/capric triglyceride | 2.00 |
| Diisostearyl malate | 1.00 |
| Squalane | 2.00 |
| Octylmethoxycinnamate | 6.00 |
| Tocopheryl acetate | 0.20 |
| Preservative | QS |
| Fragrance | OS |

### Color Extension Test Outline

1. Blend the loose powder using laboratory blender for certain minutes continuously (depending on the size of powder).
2. Measure the color of the blended loose powder obtaining L*, a* and b* values, which are assigned to L₀, a₀ and b₀.
3. Atomize the blended powder using a hammer mill with 0.020 inch screen. (1 pass)
4. Measure the color of the atomized powder thereby obtaining L*, a* and b* values, which are assigned to L₁, a₁ and b₁.
5. Repeat the process 3 and 4 obtaining L*, a* and b* values, which are assigned to Lₙ, aₙ and bₙ. (n pass)

### Evaluate Color Extension

6. Calculate ΔEₙ, which is [(Lₙ₊₁−Lₙ)²+(aₙ₊₁−aₙ)²+(bₙ₊₁−bₙ)²]^{1/2}, to determine the minimum pass number to obtain fully extended color.
7. If ΔE is less than about one (ΔE<1.0), the color is fully extended.

TABLE 8
Color extension of Formulas 1, 14 and
17, vs. Comparative Formulas 1 and 2

| | ΔE₀ | ΔE₁ | ΔE₂ | ΔE₃ | ΔE₄ |
|---|---|---|---|---|---|
| Samples | (no pulverizing) | (1 pass) | (2 pass) | (3 pass) | (4 pass) |
| Formula 1 | 0.45 | — | — | — | — |
| Formula 14 | 0.65 | — | — | — | — |
| (see Table 10) | | | | | |
| Formula 17 | 0.54 | — | — | — | — |
| (see Table 10) | | | | | |
| Comparative | 2.01 | 1.36 | 1.10 | 0.65 | — |
| Formula 1 | | | | | |
| Comparative | 2.56 | 1.65 | 1.27 | 1.10 | 0.59 |
| Formula 2 | | | | | |

TABLE 9
Showing the effect of “surface treatment,” “octinoxate,” and “titanium oxide (ultrafine)” on SPF

| | | Formula | Comparative | Comparative | Comparative |
|---|---|---|---|---|---|
| | | 18 | Formula 2 | Formula 3 | Formula 4 |
| Shade | | skin shade | skin shade | skin shade | skin shade |
| Surface Treatment | Potassium Myristate | 1.50% | — | — | — |
| | Disodium | 1.00% | — | — | — |
| | Stearoylglutamate | | | | |
| | Triethoxycaprylylsilane | 3.00% | — | — | — |
| Pigments + | Talc | 46.00% | 51.50% | 59.00% | 67.00% |
| Substrates | Mica | 22.00% | 22.00% | 22.00% | 22.00% |
| | Silica | 1.00% | 1.00% | 1.00% | 1.00% |
| | Titanium Dioxide | 8.00% | 8.00% | 8.00% | — |
| | (Ultrafine) | | | | |
| | Titanium Dioxide | 5.00% | 5.00% | 5.00% | 5.00% |
| | Yellow Iron Oxide | 2.80% | 2.80% | 2.80% | 2.80% |
| | Red Iron Oxide | 1.00% | 1.00% | 1.00% | 1.00% |
| | Black Iron Oxide | 0.20% | 0.20% | 0.20% | 0.20% |
| UV Absorbers/ | Octinoxate | 7.50% | 7.50% | — | — |
| emmolients | | | | | |
| Preservative | Phenoxyethanol | 1.00% | 1.00% | 1.00% | 1.00% |
| Total | | 100.00% | 100.00% | 100.00% | 100.00% |
| Sunscreen | in vitro SPF | 280 | 70 | 27 | 7 |
| Performance | in vivo SPF | 100 | na | na | na |
| Color Change | DE₁ (after 1 pass) | 1.3 | 2.1 | 1.2 | 1.1 |
| | DE₂ (after 2 pass) | 0.7 | 1.6 | 0.6 | 0.5 |
| | DE₃ (after 3 pass) | <0.1 | 1.2 | <0.1 | <0.1 |
| | DE₄ (after 4 pass) | na | 0.6 | na | na |
| | DE₅ (after 5 pass) | na | <0.1 | na | na |

TABLE 10
Formulas of different shades demonstrating SPF and shade extension profiles.

| | | Formula 19 | Formula 20 | Formula 21 | Formula 22 |
|---|---|---|---|---|---|
| Shade | | light pink | dark yellow | yellowish red | dark brown |
| Surface Treatment | Potassium Myristate | 1.50% | 1.50% | 1.50% | 1.50% |
| | Disodium | 1.00% | 1.00% | 1.00% | 1.00% |
| | Stearoylglutamate | | | | |
| | Triethoxycaprylylsilane | 3.00% | 3.00% | 3.00% | 3.00% |
| Pigments + | Talc | 46.00% | 45.70% | 45.37% | 37.30% |
| Substrates | Mica | 22.00% | 22.00% | 22.00% | 22.00% |
| | Silica | 1.00% | 1.00% | 1.00% | 1.00% |
| | Titanium Dioxide | 8.00% | 8.00% | 8.00% | 8.00% |
| | (Ultrafine) | | | | |
| | Titanium Dioxide | 5.00% | 0.50% | 0.50% | 0.50% |
| | Yellow Iron Oxide | 2.80% | 8.00% | 2.80% | 4.30% |
| | Red Iron Oxide | 1.00% | 0.80% | 6.00% | 4.90% |
| | Black Iron Oxide | 0.20% | 0.00% | 0.33% | 8.00% |
| UV Absorbers/ | Octinoxate | 7.50% | 7.50% | 7.50% | 7.50% |
| emmolients | | | | | |
| Preservative | Phenoxyethanol | 1.00% | 1.00% | 1.00% | 1.00% |
| Total | | 100.00% | 100.00% | 100.00% | 100.00% |
| Sunscreen | in vitro SPF | 275 | 305 | 285 | 295 |
| Performance | in vivo SPF | na | na | na | na |
| Color Change | DE₁ (after 1 pass) | 0.9 | 1.3 | 1.8 | 1.5 |
| | DE₂ (after 2 pass) | 0.2 | 0.5 | 1.0 | 0.7 |
| | DE₃ (after 3 pass) | <0.1 | <0.1 | <0.1 | <0.1 |
| | DE₄ (after 4 pass) | na | na | na | na |
| | DE₅ (after 5 pass) | na | na | na | na |

TABLE 12

| | Comparative | Formu- | Formu- | Formu- |
|---|---|---|---|---|
| Ingredient | Formula 5 | la 23 | la 24 | la 25 |
| UV-Actives | Wt % | wt % | wt % | wt % |
| Octinoxate | 3.0 | 1.3 | 3.0 | 2.9 |
| Oxybenzone | 3.0 | 1.3 | 3.0 | 2.9 |
| Cosmetic Powder | | | | |
| Uncoated silica | — | — | 1.2 | — |
| Uncoated talc | — | — | 9.8 | — |
| Coated silica | — | 1.2 | — | 1.2 |
| Coated talc | — | — | — | 9.8 |
| Remaining Ingredients | | | | |
| Hydroxyethyl Acrylate/ | 2.5 | 5.0 | 5.0 | 5.0 |
| Sodium Acryloyldimethyl | | | | |
| Taurate opolymer (and) | | | | |
| Squalane (and) Polysor- | | | | |
| bate 60 (Simulgel ™ NS) | | | | |
| Deionized water | QS 100 | QS 100 | QS 100 | QS 100 |
| Preservatives | QS | QS | QS | QS |
| Fragrance | QS | QS | QS | QS |
| Performance | | | | |
| In vitro SPF | 14 | 10 | 39 | 42 |
| In vivo SPF | 18 | NA | NA | 36 |
| In vitro SPF Index | 2.3 | 3.8 | 6.5 | 7.2 |
| In vivo SPF Index | 3.0 | NA | NA | 6.2 |
| Star Rating (UVA) | + | ++ | ++ | ++ |

The control formulation of Table 12 is typical of conventional sunscreen formulations. The control formulation (Comparative Formula 5) incorporates 6 wt % organic UV-actives and achieves an SPF value of 14 yielding an SPF Index of 2.33 (14/6). Formula 23 demonstrates that almost the same SPF value may be achieved at much lower UV-actives concentration (2.6) by including about 1% of a coated silica cosmetic powder (SPF Index 3.8). In Formulations 23 and 25, the indicated silica and talc are coated with a mixture of triethoxycaprylylsilane, aluminum myristate, and disodium stearoyl glutamate.

Formulation 24 demonstrates a 2.8-fold increase over the control SPF value yielded by 6 wt % organic UV-actives is achievable by including 11 wt % uncoated cosmetic powder. The SPF Index is increased to 6.5. By using coated cosmetic powders (Formulation 25), an increase from SPF 39 to SPF 42 is achieved and the organic UV-actives are decreased from 6 wt % to 5.8 wt % and the SPF Index is increased to 7.2.

TABLE 13

| | Formu- | Formu- | Formu- | Formu- |
|---|---|---|---|---|
| Ingredient | la 26 | la 27 | la 28 | la 29 |
| UV-Actives | | | | |
| Octinoxate | 5.8 | 5.7 | 5.7 | 5.7 |
| Cosmetic Powder Materials | | | | |
| Coated silica | 1.1 | 1.2 | 1.3 | 1.4 |
| Coated kaolin | 6.5 | 8.0 | 10.0 | 12.0 |
| Remaining Ingredients | | | | |
| Hydroxyethyl Acrylate/ | 5.0 | 5.0 | 5.0 | 5.0 |
| Sodium Acryloyldimethyl | | | | |
| Taurate opolymer (and) | | | | |
| Squalane (and) Polysor- | | | | |
| bate 60 (Simulgel ™ NS) | | | | |
| Deionized water | QS 100 | QS 100 | QS 100 | QS 100 |
| Preservatives | QS | QS | QS | QS |
| Fragrance | QS | QS | QS | QS |
| Performance | | | | |
| In vitro SPF | 20 | 21 | 23 | 23 |
| In vitro SPF Index | 3.4 | 3.7 | 4.0 | 4.0 |
| Star Rating (UVA) | + | + | + | + |
| Texture on skin | Trans- | Trans- | Trans- | Trans- |
| | parent | parent | parent | parent |

Procedure for Table 13: Oxybenzone was dissolved into Octinoxate by heating to approx. 70° C. Uncoated silica and uncoated talc were dispersed into the resulting Octinoxate solution and Hydroxyethyl Acrylate/Sodium Acryloyldimethyl Taurate opolymer (and) Squalane (and) Polysorbate 60 (Simulgel™ NS) was added. Water heated to about 70° C. was added to the powder suspended oil phase and emulsified by homogenization. Preservatives and fragrance are added to it as needed.

Formula Formulations 26 through 29 maintain constant concentrations of organic UV-actives and coated silica while providing increasing concentrations of coated kaolin substrate. Table 13 shows that those conditions cause the SPF Index to increase to a plateau. The silica and kaolin of Formulations 26 through 29 are coated with a mixture of triethoxycaprylylsilane, aluminum myristate, and disodium stearoyl glutamate.

TABLE 14

| | Formu- | Formu- | Formu- | Formu- | Formu- |
|---|---|---|---|---|---|
| Ingredient | la 30 | la 31 | la 32 | la 33 | la 34 |
| UV-Actives | | | | | |
| Octinoxate | 2.75 | 2.60 | 2.85 | 2.40 | 2.90 |
| Oxybenzone | 2.75 | 2.60 | 2.85 | 2.40 | 2.90 |
| Cosmetic Powder | | | | | |
| Materials | | | | | |
| Coated silica | 1.2 | 1.2 | 1.1 | 1.1 | 1.3 |
| Coated Beadyl ™ | 9.4 | | | | |
| Beads (aluminum calci- | | | | | |
| um sodium silicate) | | | | | |
| Coated kaolin | — | 9.0 | — | — | — |
| Coated mica | — | — | 9.8 | — | — |
| Coated titanated mica, | — | — | — | 8.2 | — |
| (Flamenco ™ Velvet) | | | | | |
| Coated talc | — | — | — | — | 10.0 |
| Remaining Ingredients | | | | | |
| hydroxyethyl acrylate/ | 4.8 | 4.8 | 4.8 | 4.8 | 4.8 |
| sodium acryloyldimethyl | | | | | |
| taurate opolymer (and) | | | | | |
| squalane (and) polysor- | | | | | |
| bate 60 (Simulgel ™ NS) | | | | | |
| Deionized water | QS 100 | QS 100 | QS 100 | QS 100 | QS 100 |
| Preservatives | QS | QS | QS | QS | QS |
| Fragrance | QS | QS | QS | QS | QS |
| Performance | | | | | |
| In vitro SPF | 38 | 42 | 42 | 56 | 42 |
| In vitro SPF Index | 6.9 | 8.1 | 7.4 | 11.7 | 7.2 |
| Star Rating (UVA) | ++ | ++ | ++ | ++ | ++ |
| Texture on skin | Trans- | Trans- | Trans- | Trans- | Trans- |
| | parent | parent | parent | parent | parent |

In Table 14, “silicate” refers to an aluminum calcium sodium silicate and the various cosmetic powder materials were coated with a mixture of triethoxycaprylylsilane, aluminum myristate, and disodium stearoyl glutamate. Flamenco Velvet refers to a mica similarly coated with a mixture of triethoxycaprylylsilane, aluminum myristate, disodium stearoyl glutamate and further with titanium dioxide. In the various formulations, the concentration of UV-actives was maintained at 5.4±0.4%, the concentration of cosmetic powder was maintained at 10.6±0.6% (1.2±0.1% silica+9.4±0.6% other cosmetic powder). The data of Table 14 show that under these conditions, the SPF, and therefore the SPF Index, strongest in Formula 33.

TABLE 15

| Ingredient | Formula 35 | Formula 36 |
|---|---|---|
| UV-Actives | | |
| Octinoxate | 2.06 | 1.64 |
| Oxybenzone | 2.06 | 0.82 |
| Avobenzone | — | 1.64 |
| Methyl anthranilate | 2.06 | — |
| Cosmetic Powder Materials | | |
| Coated silica | 1.3 | 1.1 |
| Coated kaolin | 9.6 | 7.7 |
| Remaining Ingredients | | |
| Hydroxyethyl Acrylate/Sodium | 4.8 | 4.1 |
| Acryloyldimethyl Taurate | | |
| opolymer (and) Squalane (and) | | |
| Polysorbate 60 (Simulgel ™ NS) | | |
| Deionized water | QS 100 | QS 100 |
| Disodium EDTA | — | 0.1 |
| Preservatives | QS | QS |
| Fragrance | QS | QS |
| Performance | | |
| In vitro SPF | 48 | 66 |
| In vitro SPF Index | 7.7 | 16.1 |
| Star Rating (UVA) | ++++ | ++++ |
| Texture on skin | Transparent | Transparent |

Formulations 35 and 36 show that the SPF Index may be controlled by varying the specific organic UV-active and the specific cosmetic powder substrate.

TABLE 16
Surface treated, dried powder with coated UV absorbing
oil was dispersed into “isopropyl myristate”
and was followed by adding Simugel NS and Abil WE-09.
Then a previously prepared water phase was added to the
oil phase while homogenizing at approx. 4500 rpm.

| Ingredient | Formula 37 |
|---|---|
| UV-Actives | |
| Octinoxate (Parsol MCX) | 2.25 |
| Octocrylene (Parsol 340) | 2.25 |
| Avobenzone (Parsol 1789) | 1.50 |
| Phase A (Aqueous) | |
| Deionized water | Balance to 100.00 |
| 1,3-Butylene Glycol | 2.00 |
| Sodium Chloride | 0.50 |
| Disodium EDTA | 0.10 |
| Phase B (Oil) | |
| Isopprpyl Myristate (IPM) | 17.00 |
| Polyglyceryl-4 Isostearate (and) | 5.00 |
| Cetyl PEG/PPG-10/1 Dimethicone | |
| (and) Hexyl Laurate (ABIL WE09) | |
| Hydroxyethyl Acrylate/Sodium | 3.00 |
| Acryloyldimethyl Taurate | |
| opolymer (and) Squalane (and) | |
| Polysorbate 60 (Simulgel ™ NS) | |
| Triethoxycaprylylsilane (and) | 19.0 |
| Aluminum Myristate (and) Disodium | |
| Stearoyl Glutamate (and) Kaolin (and) | |
| Mica (and) Tianium Dioxide*¹ | |
| Performance | |
| in vitro SPF | 50 |
| in vitro PFA | 15 |
| in vitro UVA/UVB ratio | 0.67 |
| in vitro Star Rating | +++ |
| in vitro SPF Index | 8.33 |
| in vivo SPF | 33 |
| in vivo PFA | 15 |

*¹Flamenco Velvet comprising a surface treatment of Triethoxycaprylylsilane (and) Aluminum Myristate (and) Disodium Stearoyl Glutamate provided onto a mixture of kaolin and titanated mica.

Formulation 37 is an example of a water-in-oil sunscreen prepared from an oven-dried wetcake. The overall treatment process and subsequent drying of the product is as described above. Phases A and B are prepared separately and then combined by homogenization. The final product is an oil-based, pourable sunscreen. In this system, the treated, dried powder is within the cosmetic oil (isopropyl myristate, “IPM”) and the sunscreen active oils remain with the treated powder due to the lipophilic nature of both the treatment and the actives. The system is stabilized with ABIL WE09 emulsifying agent.

TABLE 17
Daily-use, “tinted sunscreens.”

| | Compar- | | | |
|---|---|---|---|---|
| | ative | | | |
| | Formu- | Formu- | Formu- | Formu- |
| Ingredient | la 6 | la 38 | la 39 | la 40 |
| UV-Actives | | | | |
| Octinoxate | 0.72 | 0.72 | 0.67 | 1.56 |
| Octocrylene | 0.72 | 0.72 | 0.67 | 0.64 |
| Avobenzone | 0.72 | 0.72 | 0.23 | — |
| Cosmetic Powder Materials | | | | |
| Coated titanated mica, | — | 0.50 | 0.47 | 0.47 |
| (Flamenco Velvet) | | | | |
| Coated kaolin | — | 21.2 | 19.5 | 19.5 |
| Coated titanium doxide | — | — | — | 0.63 |
| Coated yellow iron oxide | — | — | 0.70 | 0.23 |
| pigment | | | | |
| Coated red iron oxide | — | — | 0.20 | 0.05 |
| pigment | | | | |
| Coated black iron oxide | — | — | 0.08 | 0.025 |
| pigment | | | | |
| Remaining Ingredients | | | | |
| Hydroxyethyl Acrylate/ | 3.2 | 3.2 | 2.4 | 3.2 |
| Sodium Acryloyldimethyl | | | | |
| Taurate opolymer (and) | | | | |
| Squalane (and) Polysor- | | | | |
| bate 60 (Simulgel ™ NS) | | | | |
| Isopropyl Myristate | 3.0 | 3.0 | 3.0 | 3.0 |
| Deionized water | QS 100 | QS 100 | QS 100 | QS 100 |
| Disodium EDTA | 0.1 | 0.1 | 0.1 | — |
| Preservatives | QS | QS | QS | QS |
| Fragrance | QS | QS | QS | QS |
| Performance | | | | |
| In vitro SPF | 8 | >50 | >50 | 35 |
| In vitro SPF Index | 3.7 | >20 | >30 | 15.9 |
| Star Rating (UVA) | +++ | +++ | +++ | ++ |
| Texture on skin | | Trans- | Trans- | Trans- |
| | | parent | parent | parent |
| | | | (colored) | (colored) |

Formulations 38, 39 and 40 are examples of oil-in-water sunscreens that provide both UVA and UVB protection. Each formulation was prepared by emulsifying the respective wetcake as described. In Comparative Formula 6, avobenzone comprises the UVA filter. The Comparative Formula contains the same amount of actives as Formula 38, but the Comparative Formula 6 contains no cosmetic powder. This clearly demonstrates that the use of cosmetic powder in a given formulation greatly enhances the overall SPF. It should be noted that because the value is a ratio, the UVA Star Rating (+++) is the same for both the Comparative Formula 6 and Formula 38. A much lower UVA value combined with a much lower overall SPF value may give the same UVA Star Rating (ratio) as is given by a much greater UVA value with a much larger overall SPF value. For example, 5:7 is the same ratio as 50:70 although the latter values are an order of magnitude larger. In Formula 44, the UVA protection is enhanced by employing the small primary particle size of the coated color pigments. Thus, the amount of avobenzone can be greatly reduced. It should be noted that Formula 39 is a tinted product with a reddish brown color.

Formulation 40 is free of Avobenzone. All UVA protection is derived from the primary particle size of the pigments. It is important to note that Formulas 30 through 34, and 37 also have a ++ UVA rating. However, the average UVA ratio for these samples is ˜0.37. For Formulation 40 the UVA ratio is 0.50. This underscores the fact that the pigments are providing additional UVA protection. The above final product is colored with a “skin tone” tint.

In an embodiment, a silica-based cosmetic powder is surface treated by covalently boding an organic silane. In an embodiment, the organic silane is triethoxycaprylylsilane. In an embodiment, the surface-treatment agent is a complex of triethoxycaprylylsilane, aluminum myristate, and disodium stearoyl glutamate.

TABLE 18
Triethoxycaprylylsilane-bound “Octinoxate + Octocrylene +
Avobenzxone” W/O emulsion system.

| | Comparative | |
|---|---|---|
| | Formula 7 | Formula 41 |
| Phase A | | |
| Water | 63.00 | 47.40 |
| 1,3 Butylene Glycol | 2.00 | 2.00 |
| Sodium Chloride | 0.50 | 0.50 |
| Disodium EDTA | 0.10 | 0.10 |
| Phase B | | |
| Kaolin (and) Triethoxycaprylylsilane | — | 19.00 |
| Octinoxate [Parsol MCX] | 2.25 | 2.25 |
| Octocrylene [Parsol 340] | 2.25 | 2.25 |
| Avobenzone [Parsol 1789] | 1.50 | 1.50 |
| Isopropyl Myristate | 20.40 | 17.00 |
| Polyglyceryl-4 Isostearate (and) | 5.00 | 5.00 |
| Cetyl PEG/PPG-10/1 Dimethicone | | |
| (and) Hexyl Laurate [ABIL WE09] | | |
| Hydroxyethylacrylate/Sodium | 3.00 | 3.00 |
| Axryloylmethyltaurate Copolymer | | |
| (and) Squalane (and) Polysorbate | | |
| 60 [SIMULGEL NS] | | |
| Total | 100.00 | 100.00 |
| in vitro SPF | 15 | 30 |
| in vitro PFA | 10 | 13 |
| in vitro UVA/UVB ratio | 0.63 | 0.68 |
| in vitro Star Rating | +++ | +++ |
| in vitro SPF Index | 2.5 | 5.0 |

Avobenzone was dissolved into the mixture of Octinoxate, Octocrylene, and isopropyl myristate with a propeller mixer with heating to approximately 75° C. Simulgel NS and WE-09 were added to the dissolved UV absorbents and maintained at approximately 65° C. until use. Sodium chloride, disodium EDTA and butylene glycol were dissolved in water by mild heating to approximately 50° C. in a separate vessel. The water phase was added to the oil phase while homogenizing at 4500rpm. After a few minutes mixing, the component was cooled to ambient temperature. Then, SPF performance was determined using an SPF analyzer.

Formula 41, Table 18. Triethoxycaprylylsilane treated, dried kaolin was prepared with high-speed mixer such as Henschel mixer according to procedures disclosed in U.S. Pat. No. 5,968,531, incorporated herein by reference in its entirety. The treated kaolin was dispersed into an oil phase as described above for “Comparative Formula 7.” A similarly-prepared water phase was added to the oil phase while homogenizing at 4500 rpm. After a few minutes mixing, the component was cooled down to ambient temperature. Then, SPF performance was examined with SPF analyzer.

In an embodiment, an untreated cosmetic powder is dispersed into an oil phase as described above for Comparative Formula 7. A water phase, prepared as above, is added to the oil phase with homogenization, as above. We have determined that untreated cosmetic powders, treated according to the present invention also enhances SPF performance.

What is claimed is:

1. An extended color bulk powder comprising:
at least one pigment;
at least one substrate;
at least one carboxylate surface-treatment agent;
at least one organosilane surface-treatment agent; and
at least one lipophilic UV absorber,
wherein each of the at least one surface-treatment agent is chemically immobilized to a surface of at least one of the pigments or substrates; and
wherein both the surface of the at least one pigment and the surface of the at least one substrate are treated by at least one surface-treatment agent.

2. The extended color bulk powder of claim 1, wherein the at least one carboxylate surface-treatment agent is selected from the group consisting of fatty acids and their salts, acyl amino acids and their salts, and mixtures thereof.

3. The extended color bulk powder of claim 2, wherein the fatty acid and its salt is selected from the group consisting of a straight-chained fatty acid and its salt, a branched fatty acid and its salt, an unsaturated fatty acid and its salt, and mixtures thereof.

4. The extended color bulk powder of claim 3, wherein the fatty acid and its salt has 8 to 26 carbons (C8˜C26).

5. The extended color bulk powder of claim 2, wherein the acyl amino acid and its salt is selected from the group consisting of D-acyl amino acids, L-acyl amino acids, DL-acyl amino acids, and mixtures thereof.

6. The extended color bulk powder of claim 2, wherein the acyl amino acid and its salt is selected from the group consisting of glycine, alanine, β-alanine, valine, leucine, isoleucine, phenylalanine, proline, threonine, serine, arginine, histidine, lysine, aspartic acid, glutamic acid, tyrosine, methionine, cystine, sarcosine (N-methyglycine, N-methyl-β-alanine, hydrolyzed proteins, peptides, and mixtures thereof.

7. The extended color bulk powder of claim 2, wherein the acyl amino acid and its salt comprise a fatty acid acyl derivative selected from the group consisting of unsaturated straight chain fatty acids, saturated straight chain fatty acids, unsaturated branched chain fatty acids, saturated straight chain fatty acids, and mixtures thereof.

8. The extended color bulk powder of claim 2, wherein the acyl amino acid and its salt is selected from the group consisting of sodium N-myristoyl sarcosinate, N-lauroyl-methyl-β-alanine, N-lauroyl-L-lysine, disodium N-stearoyl-L-glutamate, monosodium N-cocoyl-L-glutamate, potassium N-lauroyl-L-glutamate, triethanolamine N-cocoyl-L-glutamate, myristoyl silk amino acid, the aluminum salt of myristoyl silk amino acid, and sodium dilauramidoglutamide lysine.

9. The extended color bulk powder of claim 1, wherein the organosilane surface-treatment agent is selected from the group consisting of organopolysiloxanes, alkylsilanes, and mixtures thereof.

10. The extended color bulk powder of claim 1, wherein the lipophilic UV absorber is selected from the group consisting of 4-aminobenzoic acid (PABA), ocyldimethyl-4-aminobenzoic acid, phenylbenzimidazole sulfonic acid, cinoxate, dioxybenzone, oxybenzone, homosalate, menthyl anthranilate, octocrylene, octinoxate, octisalate, sulisobenzone, trolamine salicylate, avobenzone, terephthalyidene dicamphor sulfonic acid, 4-methylbenzylidene camphor, methylene bis-benzotriazolyl tetramethylbutylphenol, bis-ethylhexyloxyphenol methoxyphenol triazine, drometrizole trisiloxane, sodium dihydroxy dimethoxy disulfobenzophenone, octyl triazone, diethylamino hydroxybenzoyl hexyl benzoate, iscotrizinol, polysilicone-15, amiloxate, ethylhexyl dimethoxybenzylidene dioxoimidazolidine propionate, and mixtures thereof.

11. The extended color bulk powder of claim 1, wherein the pigment is substantially deagglomerated.

12. The extended color bulk powder of claim 1, wherein the pigment adheres to the substrate.

13. The extended color bulk powder of claim 1, wherein the pigment is selected from the group consisting of titanium dioxide, zinc oxide, zirconium oxide, zirconium dioxide, iron oxide, ultramarine, titanated mica, pearl pigment, manganese violet, Prussian blue, chromium oxide, chromium hydroxides, and mixtures thereof.

14. The extended color bulk powder of claim 1, wherein the substrate is selected from the group consisting of clay, mica, pearl pigment, a mica coated with titanium dioxide, talc, kaolin, sericite, silica, aluminum silicate, magnesium silicate, calcium sodium silicate, fumed silica, alumino-silicate minerals, nylon, boron nitride, an acrylate, polymethyl methacrylate (PMMA), a metal powder, ceramic powder, cotton powder, cellulose, urethane, styrene, polyolefin, polyetheylene, polyamide, zirconium, starch and starch derivatives such as aluminum starch octenylsuccinate, calcium carbonate (chalk), and mixtures thereof.

15. The extended color bulk powder of claim 1, further comprising an oil, fat, or wax.

16. The extended color bulk powder of claim 15, wherein the oil, fat, or wax is selected from the group consisting of glycerides, monoglycerides, diglycerides, triglycerides, fatty acid esters, hydroxyl acid esters, hydrocarbons, mineral oils, castor oil derivatives, vegetable-based oil esters, silicones, lipophilic vitamins, caprylic-triglyceride, capric-triglyceride, beeswax, corn oil carnauba wax, cetyloctanoate, dimethicone, diphenyldimethicone, cyclomethicone, cetyldimethicone, polysilicone-11, isostearyl neopentanoate, cetyloctanoate, diisostearyl maleate, squalane, tocopherol acetate, tocopherol, retinol, retinoic acid, isododecane, isononyl isononanoate, behenyl alcohol, cholesteryl-lauroyl glutamate, behenyl-lauroyl glutamate, octyldodecyl-lauroyl glutamate, and mixtures thereof.

17. The extended color bulk powder of claim 1, further comprising a polyglyceryl fatty acid ester, polyalkylene glycol fatty acid ester, or polyalkylene glycol alkyl ether.

18. The extended color bulk powder of claim 1, wherein the extended color bulk powder exhibits a ΔE value of less than about 2.0, as determined by the formula [(Lₙ₊₁−Lₙ)²+(aₙ₊₁−aₙ)²+(bₙ₊₁−bₙ)²]^{1/2}, after the powder is blended.

19. The extended color bulk powder of claim 1, wherein the extended color bulk powder exhibits a ΔE value of less than about 1.5, as determined by the formula [(Lₙ₊₁−Lₙ)²+(aₙ₊₁−aₙ)²+(bₙ₊₁−bₙ)₂]^{1/2}, after the powder is blended.

20. The extended color bulk powder of claim 1, wherein the extended color bulk powder has an SPF Index of at least 3.

21. The extended color bulk powder of claim 1, wherein the weight of the pigment is less than a fixed percentage of the total weight of the powder wherein the percentage is selected from the group consisting of about 70%, 60%, 50%, 40%, 30%, and 20%.

22. The extended color bulk powder of claim 1, wherein the pigment is substantially uniformly distributed onto the surface of the substrate.

23. A product comprising the extended color bulk powder of claim 1, wherein the product is selected from the group consisting of cosmetic products, makeup products, personal care products, and pharmaceutical products.

24. An extended color bulk powder, comprising at least one pigment and at least one substrate, wherein the pigment is substantially deagglomerated, wherein the pigment and substrate has a surface that has been chemically immobilized with at least one surface-treatment agent; wherein the pigment adheres to the substrate; and wherein the amount of substrate is greater than about 20% by weight of the total color bulk powder material.

25. An extended bulk powder consisting of about 65-70% substrate, about 10-25% pigment, about 3-8% surface-treatment agent, and about 3-15% oil,
wherein the substrate consists of a mixture of talc, mica, and silica;
the pigment consists of a mixture of red iron oxide, yellow iron oxide, black iron oxide, and titanium dioxide;
the oil consists of a mixture of oxinoxate and phenoxyethanol; and
the surface-treatment agent consists of a mixture of disodium stearoyl glutamate, aluminum dimyristate, and triethoxycaprylylsilane.

26. A method for preparing an extended color bulk powder, comprising:
(a) providing at least one pigment;
(b) providing at least one substrate;
(c) providing at least one UV absorber;
(d) contacting the pigment or substrate with at least one carboxylate surface-treatment agent and at least one organosilane surface-treatment agent to produce a surface-modified pigment or substrate, thereby producing a substrate to which the pigment adheres; and
(e) blending the material of step (d) until ΔE value of less than about 2.0, as determined by the formula [(Lₙ₊₁−Lₙ)²+(aₙ₊₁−aₙ)²+(bₙ₊₁−bₙ)²]^{1/2}.
